(19) United States
(12) Patent Application Publication
(10) Pub. No.: US 2022/0323401 A1
(43) Pub. Date: Oct. 13, 2022

(54) FORMULATIONS OF CANNABINOIDS FOR THE TREATMENT OF ACNE

(71) Applicants: Botanix Pharmaceuticals Ltd., Northridge (AU); Botanix Pharmaceuticals, INC., Plymouth Meeting, PA (US)

(72) Inventors: Eugene R. Cooper, Plymouth Meeting, PA (US); Matthew Callahan, Plymouth Meeting, PA (US)

(21) Appl. No.: 16/486,214

(22) PCT Filed: Jan. 24, 2018
(86) PCT No.: PCT/AU2018/050045

§ 371 Date: Aug. 15, 2019

Related U.S. Application Data
(60) Provisional application No. 62/459,313, filed on Feb. 15, 2017.

(30) Foreign Application Priority Data
Feb. 15, 2017 (AU) 2017900493

(51) Int. Cl. A61K 31/352; A61K 31/05; A61P 17/10; A61K 9/00; A61K 47/10

(52) U.S. Cl. CPC A61K 31/352; A61K 31/05; A61P 17/10; A61K 9/0014; A61K 47/10

(57) ABSTRACT

A pharmaceutical composition comprising a cannabinoid and a siloxane wherein cannabinoid is dissolved in the composition.

### TECHNICAL FIELD

The present invention relates to a pharmaceutical composition for the delivery of a cannabinoid, such as cannabidiol. The pharmaceutical composition of the present invention is particularly suited for the treatment of acne.

### BACKGROUND ART

The following discussion of the background art is intended to facilitate an understanding of the present invention only. The discussion is not an acknowledgement or admission that any of the material referred to is or was part of the common general knowledge as at the priority date of the application.

Most mammalian skin, including human skin, comprises three layers: (i) an epidermis layer, which is predominantly composed of keratinocytes and a small number of melanocytes and Langerhans cells (antigen presenting cells); (ii) a dermis layer, which contains nerve endings, sweat glands and oil (sebaceous) glands, hair follicles, and blood vessels and which is primarily composed of fibroblasts; and (iii) a hypodermis layer of deeper subcutaneous fat and connective tissue. The epidermis itself is made up of two layers, the outer stratum corneum and the inner epidermal basal layer.

Acne is a multi-factorial disease affecting the sebaceous follicle and characterized by papules, pustules, and scars. Acne affects more than 80% of 16-year old boys and girls, but is not a problem confined to teenagers. Simple attention to hygiene is no longer sufficient and antiseptic washes, so popular some years ago, are now perceived as ineffective by many sufferers and most clinicians.

During puberty, elevated androgen levels stimulate the sebaceous glands to enlarge and produce increased amounts of sebum in the sebaceous follicle. Subsequent abnormal keratinization with hyperkeratosis of the follicular epithelium leads to obstruction of the duct by horny plaque. The blocked duct becomes clogged with a dense material composed of sebum and keratinous debris forming a micro comedo, a precursor of the acne lesion. The excess sebum in the micro comedo also provides an anaerobic growth medium for Propionibacterium acnes. Lipase from the bacteria hydrolyzes sebum triglycerides into free fatty acids that are both comedogenic and pro-inflammatory. Propionibacterium acnes also secrete chemotactic factors that attract neutrophils. Lysosomal enzymes released from the neutrophils rupture the follicle wall releasing pro-inflammatory mediators, including keratin and lipids, into the surrounding dermis. Inflammatory papules appear as a result. Further inflammation with macrophages and foreign body reactions lead to cysts and nodules. The key features of the pathogenesis of acne can be characterized as: 1) increased sebum production; 2) hyper-proliferation of sebocytes (highly specialized, sebum-producing epithelial cells) that contributes to clogging of pores through which sebum is normally released to the skin surface; 3) bacterial proliferation; and 4) inflammation.

Effective management of acne can be accomplished by addressing the four key features of the pathogenesis. Topical therapy is usually the first choice for patients with mild-to-moderate inflammatory acne. The use of topical therapy minimizes potential side effects associated with the use of systemic agents. Topical therapies include benzoyl peroxide, which is the most commonly used non-prescription acne medication. It is an important antibacterial oxidizing agent that can decrease the number of Propionibacterium acnes bacteria and frequently the amount of free fatty acids. Benzoyl peroxide is the first line of monotherapy for mild acne and it is available in over-the-counter preparations. Benzoyl peroxide is applied once or twice daily and patients often experience mild redness and scaling of the skin during the first week of usage.

Tretinoin is an effective topical comedolytic agent, decreasing the cohesiveness of follicular epithelial cells and thereby inhibiting the formation of microcomedones and increasing cell turnover resulting in expulsion of existing comedones. This agent also decreases the thickness of the stratum corneum and potentiates the penetration of topical antibiotic agents. Tretinoin therapy comprises once daily application. Mild redness and peeling are a part of the therapeutic effect of the medication but can result in reduced patient compliance. Patients should be made aware that improvement may take as long as 6 to 12 weeks, and that flare-ups of acne can occur during the first few weeks of therapy. In addition, it is extremely important that patients avoid excessive exposure to the sun during treatment and comply with the designated monitoring program to deal with the well-known side effects of tretinoins.

Mild inflammatory acne lesions can also be treated with topical antibiotics including erythromycin ointment, clindamycin solution and meclocycline cream. The primary action of the antibiotics is to reduce the population of Propionibacterium acnes in the sebaceous follicle and thereby suppress the free fatty acid production. The effectiveness of topical antibiotics in the treatment of acne is limited by their low lipid solubility and subsequent difficulty in penetrating sebum-filled follicles. Topical antibiotics are applied twice daily.

Patients with moderate to severe inflammatory acne often require oral antibiotics in addition to topical therapy. The most commonly prescribed agents include tetracycline, erythromycin, minocycline and doxycycline. Treatment is usually maintained for several months. Side effects include the overgrowth of nonsusceptible organisms including Candida, which can produce vaginal and oral yeast infections.

Patients with severe inflammatory acne unresponsive to other therapy may require treatment with oral isotretinoin. Isotretinoin is a compound related to vitamin A, and is the only agent that decreases sebum production and reverses the abnormal epithelial formation process. This agent can also decrease the population of Propionibacterium acnes in the sebaceous follicle. Duration of therapy is usually 20 weeks and the satisfactory response rate is quite high. However, treatment is often accompanied by many side effects, including dry skin, pruritus, epistaxis and photosensitivity, as well as hypertriglyceridemia, abnormal liver function tests, electrolyte imbalances and elevated platelet counts. Most serious though, is the teratogenic effect of isotretinoin. Use of isotretinoin during pregnancy is absolutely contraindicated. So serious is the potential for death or teratogenic effects to a foetus, isotretinoin is practically contraindicated in women of child-bearing age. Use of isotretinoin must be accompanied by a guarantee by the patient that conception will be avoided at any and all costs.

Because acne is a multi-factorial disease which is manifest to varying degrees, it is important for the physician to assess the patient to attempt to find therapies which will be helpful to the patient without causing major side effects. All of the current conventional treatments are associated with some degree of adverse side effects that limit their usefulness.

It is against this background that the present invention has been developed.

The present invention seeks to provide a composition and method to reduce the effects of acne, or to provide the consumer with a useful or commercial choice.

### SUMMARY OF INVENTION

In accordance with the present invention, there is provided a pharmaceutical composition comprising a cannabinoid and a siloxane wherein the cannabinoid is dissolved in the composition. In accordance with one embodiment, the cannabinoid is cannabidiol. In accordance with another aspect of the invention, the pharmaceutical composition is a topical pharmaceutical composition. The siloxane forms a volatile solvent for the cannabinoid.

The cannabinoids delivered by the present invention preferably penetrate into the epidermis of the skin, and most of the cannabinoids remain in that layer. Preferably some further penetrates to the dermis and some cannabinoid penetrates further into the hypodermal layer, to be absorbed systemically. The skin to which the composition is delivered is preferably mammalian skin, more preferably human mammalian skin.

The compositions of the invention may further contain (i) further volatile solvents such as low molecular weight alcohols, and/or (ii) less volatile solvents such as fatty alcohols and/or alkyl polypropylene glycol/polyethylene glycol ethers (alkyl PEG/PPG ethers). The less volatile solvent is called the residual solvent as it may remain on the skin after evaporation of the siloxane (and evaporation of the further volatile solvent if it is present) These additional volatile and residual solvent excipients may further enhance the capacity of the compositions of the invention to produce concentrated cannabinoid solutions in situ, and/or facilitate the delivery of the cannabinoid to the epidermis and the dermis for the treatment of acne.

In accordance with the present invention, there is provided a method for treating or preventing acne in a patient in need of such treatment, the method comprising topically administering a prophylactically or therapeutically effective amount of pharmaceutical composition according to the invention.

In accordance with the present invention, there is provided a method for use of a cannabinoid and a siloxane for the manufacture of a pharmaceutical composition for the prevention or treatment of acne in a patient in need thereof, wherein the cannabinoid is dissolved in the composition.

In accordance with the present invention, there is provided a method for use of a topical composition according to the invention for the prevention or treatment of acne.

In one embodiment, the pharmaceutical composition is a topical composition.

### DESCRIPTION OF THE FIGURES

FIG. 1: Graphical representation of the mean plasma CBD concentrations on Day 1 (Linear Scale).

FIG. 2: Graphical representation of the mean plasma CBD concentrations on Day 21 (Linear Scale).

FIG. 3: Graphical representation of the data shown in Table 11 for delivered CBD. Data is shown in μg/cm². A Dixon's Qtest with 95% confidence was first run on the data to identify and remove outliers.

FIG. 4: Graphical representation of the data shown in Table 11 for delivered CBD. Data is shown in μg/cm². A Dixon's Qtest with 95% confidence was first run on the data to identify and remove outliers.

FIG. 5: Graphical representation of the data shown in Table 12 for delivered CBD. Data is shown in percent delivery. A Dixon's Qtest with 95% confidence was first run on the data sets to identify and remove outliers.

FIG. 6: Graphical representation of the data shown in Table 12 for delivered CBD. Data is shown in percent delivery. A Dixon's Qtest with 95% confidence was first run on the data sets to identify and remove outliers.

FIG. 7: Graphical representation of the data shown in Table 13 for delivered CBD. Data is shown in percent delivery. A Dixon's Qtest with 95% confidence was first run on the data sets to identify and remove outliers.

FIG. 8: Graphical representation of data shown in Table 14 for CBD delivered into the skin. Data is shown in μg/g tissue. A Dixon's Qtest with 95% confidence was first run on the data to identify and remove outliers.

### DETAILED DESCRIPTION OF THE INVENTION

### The Endocannabinoid System (ECS), Cannabinoids, Cannabidiol and Acne

Identification of the main cannabinoid receptors (CB1 and CB2), their endogenous lipid ligands (endocannabinoids), biosynthetic pathways and metabolizing enzymes (collectively termed the ECS), coupled with the discovery and/or rational design of numerous exogenous ligands for CB receptors, has triggered an exponential growth in studies exploring the continuously growing regulatory functions of this newly discovered physiological system both in health and disease.

Modulating the activity of the ECS holds therapeutic potential for a multitude of diseases and pathological conditions affecting humans, ranging from inflammatory, neurodegenerative, gastrointestinal, liver, cardiovascular disorders and obesity, to ischemia/reperfusion injury, cancer and pain.

The most extensively studied endocannabinoids are anandamide (N arachidonoylethanolamine, AEA) and 2-arachidonoylglycerol (2-AG). Multiple pathways are involved in synthesis and cellular uptake of these lipid mediators. The most common degradation pathways for AEA and 2-AG are the fatty acid amid hydrolase (FAAH) and monoacylglycerol lipase (MAGL) enzyme. Endocannabinoids, similar to Δ⁹-tetrahydrocannabinol (THC; the main active ingredient of the plant Cannabis sativa), predominantly exert their physiological effects via two main G-protein-coupled cannabinoid receptors; however, numerous additional signalling mechanisms and receptor systems (e.g. transient receptor potential cation channel, subfamily V, member 1; TRPV1) might also be involved. Initially, the CB1-mediated effects were described centrally and CB1 receptors were thought to be restricted to the central nervous system, whereas CB2 was first identified at the periphery in immune cells.

Unfortunately, due to its highly lipophilic nature, cannabinoids such as cannabidiol are poorly absorbed through membranes such as the skin. Therefore, the success of administering therapeutically effective quantities of a cannabinoid such as cannabidiol to a mammal in need thereof within a reasonable time frame and over a suitable surface area has been substantially limited.

CBD may play a beneficial role in decreasing unwanted skin cell growth, sebum production and skin inflammation associated with many human skin diseases.

It is considered that CBD may:
normalise excessive lipid synthesis of human sebocytes (the cells from the oil producing sebaceous glands in the skin which disintegrate and release their oil content);
decrease proliferation (but not the viability) of these human sebocytes;
inhibit hyperproliferation of keratinocytes; and
exert universal anti-inflammatory actions.

Without being held to any theory, we believe that the mode of action of CBD for anti-acne activity involves the suppression of mediators of inflammatory responses. CBD has been shown to have lipostatic, anti-proliferative, and anti-inflammatory effects on immortalized human sebocytes. There is a physiological regulatory function of the endocannabinoid system (ECS) in proliferation, differentiation, apoptosis and cytokine, mediator and hormone production of various cell types of the skin and appendages (e.g. hair follicle, sebaceous gland), and there is evidence on the putative involvement of the ECS in certain pathological conditions of the skin including acne and seborrhea [Biro, 2009].

In vitro studies have shown CBD to stimulate the human vanilloid receptor type 1 (VR1) using HEK-hVR1 transfected cells with a maximum effect similar in efficacy to that of capsaicin, and to inhibit anandamide (an endogenous CBD neurotransmitter) using rat basophilic leukemia cells [Bisogno 2001, Mechoulam 2002]. These findings have suggested a mode of action for the anti-inflammatory properties of CBD. In vivo studies with intravenous (i.v.) administration of CBD (1 mg/kg) attenuated ovalbumin-induced airway obstruction in sensitized guinea-pigs, indicating a potential role of CBD in reducing immune-induced inflammatory reactions [Dudasova 2013]. Similarly, CBD (5 mg/kg, i.v.) given to rats once daily for 4 weeks attenuated cardiac inflammation produced by doxorubicin [Fouada 2013].

### Composition

The present invention is based on the surprising discovery that a cannabinoid, such as cannabidiol, can be dissolved in a siloxane to form a pharmaceutical composition. Further, that pharmaceutical composition may be topically applied, after which at least some the siloxane evaporates to concentrate the cannabinoid in situ, facilitating permeation to the therapeutically relevant regions of the skin (preferably the epidermis and dermal layer) for the treatment of acne.

There is therefore provided a pharmaceutical composition comprising a cannabinoid and a siloxane wherein the cannabinoid is dissolved in the composition. In accordance with one embodiment, the cannabinoid is cannabidiol. In accordance with another aspect of the invention, the pharmaceutical composition is a topical pharmaceutical composition. The siloxane forms a volatile solvent for the cannabinoid.

Unless the context requires otherwise, the term ‘acne’, as used herein, means one or more of: acne vulgaris, neonatal and infantile acne, perioral dermatitis, acne conglobata, hidradenitis suppurative, acne filminans, pyoderma faciale, acne excoriee des jeunes filles, acne mechanica, acne tropicalis, acne aestivalis, favre-racouchot syndrome, drug-induced acne, acne cosmetica, pomade acne, occupational acne, chloracne, steroid acne, rosacea, acne keloidalis nuchae and gram-negative folliculitis.

High concentrations of dissolved cannabinoids, including cannabidiol (as opposed to solid cannabinoids) are expected to be advantageous in terms of enhancing the relevant extent of delivery into the skin, particularly the epidermis (including the epidermal basal layer), with some penetration into the dermis. It is thought that the high concentration of dissolved cannabinoids on the outer surface of the skin causes a concentration gradient that enhances penetration of the cannabinoid into the skin, particularly the epidermis and the dermis.

In order to achieve local distribution for the treatment of acne, it is advantageous for the majority of the cannabinoid, such as cannabidiol, to penetrate into the epidermis and preferably remain there, and for some cannabinoid to further penetrate to the dermis and the hypodermal layer to be absorbed systemically. In such a case, the cannabidiol would concentrate mainly in the epidermis, thus maximizing its local effect. Not only does the localized effect increase the potential therapeutic benefit, it potentially lessens the frequency and severity of any potential side-effects associated with systemic cannabinoid administration, because the amount of active compound circulating in the patient is reduced.

In one preferred embodiment, the composition is non-aqueous. In another preferred embodiment, the composition does not comprise a preservative.

The present invention is based at least in part on the surprising discovery that cannabinoids can be topically administered as (i) concentrated solutions of cannabinoid in siloxane, or (ii) suspensions of crystalline cannabinoids in concentrated solutions of cannabinoid in siloxane. In either case, the preferred cannabinoid is cannabinol. The compositions of the present invention may form a highly concentrated, non-crystalline, thin layer of a cannabinoid on the skin surface, after partial or complete evaporation of the volatile siloxane, and without crystallization of the cannabinoid.

By using the volatile solvent siloxane, one can achieve much higher, non-crystalline (i.e., in solution), concentrations of cannabinoids. The cannabinoids can be dissolved in much higher concentrations of the volatile solvent siloxane than many other less volatile solvents, and then once applied to the skin and the volatile siloxane has evaporated, the cannabinoids remain on the skin in high concentrations.

The cannabinoids are preferably kept in a non-crystalline form on the skin after evaporation of the siloxane by the addition of a less volatile solvent than siloxane. This less volatile solvent is called the residual solvent, as it may remain on the skin after evaporation of the volatile solvent (siloxane and optionally another volatile solvent such as a low molecular weight alcohol) to keep the cannabinoid in a non-crystalline state after evaporation of the siloxane. Preferably the residual solvent is an alkyl polypropylene glycol/polyethylene glycol ether and/or a fatty acid alcohol. Preferably the residual solvent has a low volatility such that less than 5% would evaporate at skin temperature over 24 hours. Preferably, the residual solvent has a chain structure that has a hydrophobic end and a hydrophilic end. Preferably the residual solvent is a liquid at or below 32° C. Preferably the residual solvent dissolves siloxane. Preferably the residual solvent maintains the cannabinoid in non-crystalline form in concentrations of 20% up to 70% cannabinoid.

The total amount of the volatile solvent (siloxane and optionally another volatile solvent such as a low molecular weight alcohol), and the residual solvent if present, required is sufficient to keep the cannabinoid non-crystalline at room temperature for between about 2-8 hours once the composition is applied to the skin.

TABLE 1
Concentration of CBD on skin after evaporation of volatile solvents

| | | | | Final CBD concen- |
|---|---|---|---|---|
| | Initial | | | tration in residual |
| | CBD | | | solvent(s) after |
| | Concen- | Volatile | Residual | evaporation of |
| Formu- | tration | Component(s) | solvent(s) | volatile component(s) |
| lation | % w/w | % w/w | % w/w | % w/w |
| 1 | 0.1 | 99.7 | 0.2 | 33.3 |
| 2 | 0.5 | 99.3 | 0.2 | 71.4 |
| 3 | 1.0 | 98.8 | 0.2 | 83.3 |
| 4 | 1.0 | 98.0 | 1.0 | 50.0 |
| 5 | 5.0 | 94.0 | 1.0 | 83.3 |
| 6 | 10.0 | 89.0 | 1.0 | 90.9 |
| 7 | 1.0 | 97.0 | 2.0 | 33.3 |
| 8 | 5.0 | 93.0 | 2.0 | 71.4 |
| 9 | 10.0 | 88.0 | 2.0 | 83.3 |
| 10 | 1.0 | 96.0 | 3.0 | 25.0 |
| 11 | 5.0 | 92.0 | 3.0 | 60.0 |
| 12 | 10.0 | 87.0 | 3.0 | 76.9 |

Such administration is expected to result in enhanced delivery of a cannabinoid, such as cannabidiol, to the epidermis and dermis of the skin, which is expected to be effective in significantly reducing, and therefore, treating acne in patients in need of such treatment.

In addition to enhanced delivery, the present invention may allow larger doses of cannabinoids, such as cannabidiol, to be applied without having to have a thick layer of residue that would be rubbed off or be unacceptable to the user. The topical pharmaceutical compositions of the present invention allow more rapid delivery of the cannabinoid due to the metastable high driving force or supersaturation of the composition. In summary, it is thought that the high concentration of dissolved cannabinoids on the outer surface of the skin causes a concentration gradient that enhances penetration of the cannabinoid into the epidermis and dermis.

Therefore, in one aspect, the present invention comprises a topical composition comprising a solution of a cannabinoid in a siloxane. In one embodiment, the cannabinoid is cannabidiol.

The preferred ratio of cannabinoid to siloxane to residual solvent is selected from the range consisting of (w/w %): 0.5-20% cannabinoid, between 1-99% siloxane and between 0.1-99% residual solvent; between 5-20% cannabinoid, between 4-70% siloxane and between 1%-70% residual solvent; between 1-15% cannabinoid, between 20-95% siloxane and between 1-15% residual solvent.

Definitions: CBD: cannabidiol (CPD), IPA: isopropyl alcohol, MO: occlusive mineral oil (a viscous liquid petrolatum), HDS: hexylmethyldisiloxane, PMS: polymethylsiloxane 10⁶ cSt, HDA: 2-hexyldecyl alcohol, PG: propylene glycol, OA: oleyl alcohol, EtOH: ethanol, ODDA: octyldodecyl alcohol, AE: arlamol E, IPA: isopropyl alcohol and Klucel MF: hydroxypropylcellulose (brand name Klucel® MF from Ashland, Inc.).

In one preferred embodiment, the composition is selected from the group consisting of (w/w %):
5% CBD/10% OA/10% PG/10% HDS/65% IPA
14% CBD/9% OA/9% PG/9% HDS/59% IPA
14% CBD/4.5% OA/13.5% PG/4.5% HDS/63.5% IPA
15% CBD/5% PMS/10% OA/70% HDS
15% CBD/10% argan oil/10% HDS/65% IPA
10% CBD/7% argan oil/7% ISA/9% PMS/67% HDS
15% CBD/13% IPA/7% PMS/66% HDS
15% CBD/12.5% HDA/6% PMS/66.5% HDS
15% CBD/12.5% ODDA/6% PMS/66.5% HDS
15% CBD/10% HDA/40% IPA/35% HDS
15% CBD/10% ODDA/40% IPA/35% HDS
7.2% CBD/6.3% PMS/1.4% MO/1.8% IPA/83.3% HDS
20% CBD/10% ODDA/70% IPA
9.5 CBD/4.8% ODDA/57.1% EtOH/28.6% HDS
10% CBD/12.5% PMS/4.5% IPA/72% HDS
5% CBD/2.5% HDA/50% IPA/41% HDS/1% KlucelMF
5% CBD/3.33% HDA/50% IPA/40.67% HDS/1% KlucelMF
5% CBD/3.33% HDA/75% IPA/15.67% HDS/1% KlucelMF
10% CBD/6.67% HDA/75% IPA/7.33% HDS/1% KlucelMF
15% CBD/10% HDA/70% IPA/4% HDS/1% KlucelMF
15% CBD/7.5% HDA/70% IPA/6% HDS/1.5% KlucelMF
5% CBD/2.5% HDA/1% PMS/91.5% HDS
10% CBD/5% HDA/1% PMS/84% HDS
15% CBD/7.5% HDA/1% PMS/1% IPA/1% D5/74.5% HDS
5% CBD/2% AE/1% PMS/92% HDS
10% CBD/4% AE/1% PMS/1% IPA/84% HDS
5% CBD/2.5% HDA/1% PMS/91.5% HDS
5% CBD/1.7% HDA/1.2% PMS/92.1% HDS
5.25% CBD/1.15% PMS/1.22% IPA/92.38% HDS
5% CBD/2.5% AE/1% PMS/91.5% HDS
5% CBD/1% AE/1% PMS/93% HDS
5% CBD/2.5% IPM/1% PMS/1% IPA/90.5% HDS
10% CBD/4% AE/1% PMS/1% IPA/84% HDS
5% CBD/2% AE/1% PMS/92% HDS
5% CBD/2.5% HDA/5% PMS/87.5% HDS
10% CBD/6.67% HDA/5% PMS/78.33% HDS
15% CBD/7.5% HDA/5% PMS/1% IPA/71.5% HDS
15% CBD/7.5% HDA/10% PMS/1% IPA/66.5% HDS

In a further preferred embodiment, the composition is selected from the group consisting of:
5% CBD/3.33% HDA/50% IPA/40.67% HDS/1% KlucelMF
5% CBD/3.33% HDA/75% IPA/15.67% HDS/1% KlucelMF
10% CBD/6.67% HDA/75% IPA/7.33% HDS/1% KlucelMF
15% CBD/10% HDA/70% IPA/4% HDS/1% KlucelMF
15% CBD/7.5% HDA/70% IPA/6% HDS/1.5% KlucelMF
5% CBD/2% AE/1% PMS/92% HDS
10% CBD/4% AE/1% PMS/1% IPA/84% HDS
5% CBD/2.5% HDA/1% PMS/91.5% HDS
10% CBD/5% HDA/1% PMS/84% HDS
15% CBD/7.5% HDA/1% PMS/1% IPA/1% D5/74.5% HDS
5% CBD/1.7% HDA/1.2% PMS/92.1% HDS
5.25% CBD/1.15% PMS/1.22% IPA/92.38% HDS

In one preferred embodiment, the following formulations are solutions: 5% CBD/10% OA/10% PG/10% HDS/65% IPA, 14% CBD/9% OA/9% PG/9% HDS/59% IPA, 14% CBD/4.5% OA/13.5% PG/4.5% HDS/63.5% IPA, 5% CBD/2% AE/1% PMS/92% HDS. In another preferred embodiment, these formulations are gelled with 1% Klucel.

In one preferred form, the composition is a gel. In another preferred form, the composition is a spray. The composition may or may not contain water. Preferably, the composition does not contain water, i.e. it is non-aqueous.

### Siloxane

Siloxanes do not burn, sting or have an odour, and thus are highly advantageous for topical application for the treatment of acne. Importantly for the compositions of the present invention, siloxanes, due to their low molecular weight, are highly volatile.

In one embodiment, the siloxane contains two or three silicon atoms. The siloxanes may have between one and eight methyl groups. In one embodiment, the siloxane is selected from the group consisting of: hexamethyldisiloxane, octamethyltrisiloxane and combinations thereof. These are the most volatile siloxanes, and are thus the most advantageous. Preferably the level of volatility of the siloxane is about the same as that of isopropyl alcohol.

In another embodiment, the siloxane contains 4 or 5 silicon atoms, and is, for example, decamethyltetrasiloxane or dodecamethylpentasiloxane. In another embodiment, the siloxane is a cyclical 4 or 5 silicon atom compound such octamethylcyclotetrasiloxane (CAS #556-67-2) or decamethylcyclopentasiloxane (CAS #541-02-6).

In certain embodiments, further improvements in the solubility and crystallinity characteristics of the cannabinoid in the siloxane may be achieved by the addition of a further volatile solvent in the form of an alcohol, including a low molecular weight alcohol. An improvement in the solubility and crystallinity characteristics of the cannabinoid in the siloxane may also be achieved by the addition of an alkyl PEG/PPG ether and/or a fatty alcohol.

### Alkyl Polypropylene Glycol/Polyethylene Glycol Ethers

In certain embodiments, further improvements in the solubility characteristics of the cannabinoid, such as cannabidiol, in the siloxane may be achieved by the addition of alkyl polypropylene glycol/polyethylene glycol ethers (alkyl PEG/PPG ethers). The properties of alkyl PEG/PPG ethers, as well as suitable alkyl PEG/PPG ethers that can be used in accordance with this invention, are discussed in the Cosmetic Ingredient Review (CIR) Expert Panel 2013 “Safety Assessment of Alkyl PEG/PPG Ethers as Used in Cosmetics” Report (www.cir-safety.org/sites/default/files/PEGPPG062013tent.pdf; accessed 21 Dec. 2016) and the contents of that document are incorporated herein.

The alkyl PEG/PPG ethers also act as a residual solvent to assist in maintaining the cannabinoid in a non-crystalline state after evaporation of some or all of the siloxane and the optional low molecular weight alcohol.

Advantageously, in some embodiments, the composition also comprises one or more alkyl PEG/PPG ethers. Alkyl PEG/PPG ethers are the reaction products of an alkyl alcohol and one or more equivalents each of ethylene oxide and propylene oxide (forming repeats of polyethylene glycol (PEG) and polypropylene glycol (PPG), respectively).

The inventors have found that the addition of alkyl PEG/PPG ethers, including polypropylene glycol ethers of stearyl alcohol and butyl alcohol, can improve the solubility of cannabinoids, such as cannabidiol, in siloxane solvents. This ability to increase the concentration of the cannabinoid in the initial composition and in the final composition on the skin after application and evaporation makes it possible to achieve high residual concentrations of cannabinoids on the skin. The alkyl PEG/PPG ethers provide a residual solvent that can retain the cannabinoid in solution at an exceptionally high concentration after evaporation of the volatile solvent or solvent mixture.

Advantageously, in some embodiments, the alkyl PEG/PPG ethers are liquids at ambient temperatures. Preferably the alkyl PEG/PPG ethers are liquids at about 30° C., or less, or at about 25° C.

Advantageously, in some embodiments, the alkyl PEG/PPG ethers have a low volatility such that less than 5% would evaporate at skin temperature over 24 hours.

Advantageously, in some embodiments, the alkyl PEG/PPG ether has a PEG/PPG chain length of between 10-50 PG units and an ether component of between 2-20 carbons, wherein the sum of the PG units and the carbons of the ether component is preferably between 20 and 60. A range of alkyl PEG/PPG ethers are discussed in the Cosmetic Ingredient Review (CIR) Expert Panel 2013 “Safety Assessment of Alkyl PEG/PPG Ethers as Used in Cosmetics” Report (www.cir-safety.org/sites/default/files/PEGPPG062013tent.pdf; accessed 21 Dec. 2016) and the contents of that document are incorporated herein.

Advantageously, in some embodiments, the alkyl PEG/PPG ether is selected from the group consisting of: polypropylene glycol ethers of stearyl alcohol or butyl alcohol and combinations thereof.

In specific embodiments, the alkyl PEG/PPG stearyl ether or butyl ether is selected from the group consisting of: polypropylene glycol (PPG) stearyl ethers and polypropylene glycol butyl ethers such as PPG-15 stearyl ether and PPG-40 butyl ether and combinations thereof.

In specific embodiments, the relative amount of alkyl PEG/PPG ether is selected from the following group; at least 1% w/w, at least 2% w/w, at least 3% w/w, at least 4% w/w, at least 5% w/w. In specific embodiments, the maximum concentration of the alkyl PEG/PPG ether is 50% w/w. In specific embodiments, the maximum concentration of the alkyl PEG/PPG ether is 80% w/w.

Preferably the amount of alkyl PEG/PPG ether is sufficient to keep the cannabinoid is a non-crystalline form on the skin after partial or complete evaporation of the more volatile solvent or solvents.

### Low Molecular Weight Alcohol

Advantageously, in some embodiments, the topical composition also comprises a low molecular weight alcohol. The inventors have found that small amounts of a low molecular weight alcohol may improve the solubility of cannabinoids, such as cannabidiol, in siloxane solvents. This ability to increase the concentration of the cannabinoid in the initial composition makes it possible to achieve high residual concentrations of cannabinoids on the skin after application. Preferably the low molecular weight alcohol forms a further volatile solvent in addition to the siloxane. Preferably the level of volatility of the low molecular weight alcohol is about the same as that of isopropyl alcohol. The addition of a further volatile solvent such as a low molecular weight alcohol may be of particular advantage if the concentration of cannabinoid in the initial composition is very high.

Advantageously, in some embodiments, the low molecular weight alcohol is a liquid at ambient temperatures. Preferably the low molecular weight alcohol is liquid at about 30° C., or less, or at about 25° C. Preferably the level of volatility of the low molecular weight alcohol is about the same as that of isopropyl alcohol.

Advantageously, in some embodiments, the low molecular weight alcohol is selected from the group consisting of: C₂₋₆ alcohols, and combinations thereof. Advantageously, in some embodiments, the low molecular weight alcohol is selected from the group consisting of: C₂₋₄ alcohols, and combinations thereof.

In specific embodiments, the low molecular weight alcohol is selected from the group consisting of: ethyl alcohol (or ethanol), n-propanol, isopropyl alcohol, butanol and combinations thereof.

In specific embodiments, the relative amount of low molecular weight alcohol selected from the following group: at least 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 20% w/w, 25% w/w, 30% w/w, 35% w/w, 40% w/w, 45% w/w. In specific embodiments, the maximum concentration of the low molecular weight alcohol is 50% w/w. In specific embodiments, the maximum concentration of the low molecular weight alcohol is 60% w/w, 70% w/w, 80% w/w. The amount of low molecular weight alcohol may be between 1% w/w and 50% w/w, 1% w/w and 40%, 1% w/w and 30% w/w, 1% w/w and 20% w/w, 1% w/w and 10% w/w.

### Fatty Alcohol

Advantageously, in certain embodiments, the topical composition is further characterised in that the composition comprises a fatty alcohol. The purpose of the fatty alcohol is to act as a solvent for the cannabinoid once the volatile components, such as the siloxane and, optionally, the low molecular weight alcohol, have evaporated. In specific embodiments the fatty alcohol is a C₁₂₋₂₂ fatty alcohol. In specific embodiments, the fatty alcohol is a C₁₆₋₂₂ fatty alcohol. In specific embodiments, the fatty alcohol is selected from the group consisting of: oleyl alcohol, isostearyl alcohol, octyldodecyl alcohol, 2-hexyl decyl alcohol.

In specific embodiments, the relative amount of fatty alcohol selected from the following group; at least 2% w/w, at least 3% w/w, at least 4% w/w, at least 5% w/w. In specific embodiments, the maximum concentration of the fatty alcohol is 50% w/w. In specific embodiments, the maximum concentration of the fatty alcohol is 80% w/w.

Preferably the amount of fatty alcohol is sufficient to keep the cannabinoid is a non-crystalline form on the skin after partial or complete evaporation of the more volatile solvent or solvents.

### Cannabinoid

Preferably, the cannabinoid is cannabinol. Alternatively, the cannabinoid is any compound that interacts with the cannabinoid receptor. This may include various cannabinoid mimetics, such as certain tetrahydropyran analogs (e.g., Δ9-tetrahydrocannabinol, Δ8-tetrahydro-cannabinol, 6,6,9-trimethyl-3-pentyl-6H-dibenzo [b,d]pyran-1-ol, 3-(1, 1-dimethylheptyl)-6, 6a, 7, 8, 10, 10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylheptyl,(+)-(3S,4S)-7-hydroxy-Δ6-11-hydroxy-Δ9-tetrahydrocannabinol, and Δ8-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate)), certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone); certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxpropyl)-1′,2′,3′,4′,5′,6′-hexahydrobiphenyl); cannabinol; cannbigerol; tetrahydrocannabivarin; cammabidvarin; cannabichromene; and includes synthetic cannabinoids (such as nabilone, rimonabant, JWH-018, JWH-073, CP-55940, dimethylheptlpryan, HU-210, HU-331, SR144528, WIN 55,212-2, JWH-133, Levonantradol, AM-2201) as well as salts and analogs thereof.

In certain embodiments, the concentration of cannabinoid in the topical composition of the invention may be selected from the group consisting of: at least 2% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, and at least 15% w/w.

In certain embodiments, the concentration of cannabinoid in the topical composition may be selected from the group consisting of: at least 20% w/w, at least 30% w/w at least 40% w/w, at least 50% w/w, at least 60% w/w, at least 65% w/w, at least 70% w/w, at least 80% w/w, at least 90% w/w, at least 95% w/w and at least 99% w/w. Such concentrations may be achieved after at least partial evaporation of the volatile siloxane and, optionally, low molecular weight alcohol components.

In certain embodiments, the concentration of cannabinoid in the topical composition may be within a range with a lower limit selected from the group consisting of: 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, and 15% w/w;

and an upper limit selected from the group consisting of:
20% w/w, 30% w/w, 40% w/w, 50% w/w, 60% w/w, 65% w/w, 70% w/w, 80% w/w, 90% w/w, 95% w/w, and 99% w/w.

In certain embodiments, the concentration of the cannabinoid in the topical composition may be within a range selected from the group consisting of:

1% w/w, 2% w/w to 99% w/w, 3% w/w to 70% w/w, 4% w/w to 70% w/w, 5% w/w to 70% w/w, 6% w/w to 70% w/w, 7% w/w to 70% w/w, 8% w/w to 99% w/w, 9% w/w to 99% w/w, 10% w/w to 99% w/w, 11% w/w to 99% w/w, 12% w/w to 99% w/w, 13% w/w to 99% w/w, 14% w/w to 99% w/w, and 15% w/w to 99% w/w.

In certain embodiments, the concentration of the cannabinoid in the topical composition may be within a range selected from the group consisting of:

1% w/w, 2% w/w to 95% w/w, 3% w/w to 95% w/w, 4% w/w to 95% w/w, 5% w/w to 95% w/w, 6% w/w to 95% w/w, 7% w/w to 95% w/w, 8% w/w to 95% w/w, 9% w/w to 95% w/w, 10% w/w to 95% w/w, 11% w/w to 95% w/w, 12% w/w to 95% w/w, 13% w/w to 95% w/w, 14% w/w to 95% w/w, and 15% w/w to 95% w/w.

In certain embodiments, the concentration of the cannabinoid in the topical composition may be within a range selected from the group consisting of:

1% w/w, 2% w/w to 90% w/w, 3% w/w to 90% w/w, 4% w/w to 90% w/w, 5% w/w to 90% w/w, 6% w/w to 90% w/w, 7% w/w to 90% w/w, 8% w/w to 90% w/w, 9% w/w to 90% w/w, 10% w/w to 90% w/w, 11% w/w to 90% w/w, 12% w/w to 90% w/w, 13% w/w to 90% w/w, 14% w/w to 90% w/w, and 15% w/w to 90% w/w.

In certain embodiments, the concentration of the cannabinoid in the topical composition may be within a range selected from the group consisting of:

1% w/w, 2% w/w to 80% w/w, 3% w/w to 80% w/w, 4% w/w to 80% w/w, 5% w/w to 80% w/w, 6% w/w to 80% w/w, 7% w/w to 80% w/w, 8% w/w to 80% w/w, 9% w/w to 80% w/w, 10% w/w to 80% w/w, 11% w/w to 80% w/w, 12% w/w to 80% w/w, 13% w/w to 80% w/w, 14% w/w to 80% w/w, and 15% w/w to 80% w/w.

In certain embodiments, the concentration of the cannabinoid in the topical composition may be within a range selected from the group consisting of:

1% w/w, 2% w/w to 70% w/w, 3% w/w to 70% w/w, 4% w/w to 70% w/w, 5% w/w to 70% w/w, 6% w/w to 70% w/w, 7% w/w to 70% w/w, 8% w/w to 70% w/w, 9% w/w to 70% w/w, 10% w/w to 70% w/w, 11% w/w to 70% w/w, 12% w/w to 70% w/w, 13% w/w to 70% w/w, 14% w/w to 70% w/w, and 15% w/w to 70% w/w.

In certain embodiments, the concentration of the cannabinoid in the topical composition may be within a range selected from the group consisting of:

1% w/w, 2% w/w to 65% w/w, 3% w/w to 65% w/w, 4% w/w to 65% w/w, 5% w/w to 65% w/w, 6% w/w to 65% w/w, 7% w/w to 65% w/w, 8% w/w to 65% w/w, 9% w/w to 65% w/w, 10% w/w to 65% w/w, 11% w/w to 65% w/w, 12% w/w to 65% w/w, 13% w/w to 65% w/w, 14% w/w to 65% w/w, and 15% w/w to 65% w/w.

In certain embodiments, the concentration of the cannabinoid in the topical composition may be within a range selected from the group consisting of:

1% w/w, 2% w/w to 60% w/w, 3% w/w to 60% w/w, 4% w/w to 60% w/w, 5% w/w to 60% w/w, 6% w/w to 60% w/w, 7% w/w to 60% w/w, 8% w/w to 60% w/w, 9% w/w to 60% w/w, 10% w/w to 60% w/w, 11% w/w to 60% w/w, 12% w/w to 60% w/w, 13% w/w to 60% w/w, 14% w/w to 60% w/w, and 15% w/w to 60% w/w.

In certain embodiments, the concentration of the cannabinoid in the topical composition may be within a range selected from the group consisting of:

1% w/w, 2% w/w to 50% w/w, 3% w/w to 50% w/w, 4% w/w to 50% w/w, 5% w/w to 50% w/w, 6% w/w to 50% w/w, 7% w/w to 50% w/w, 8% w/w to 50% w/w, 9% w/w to 50% w/w, 10% w/w to 50% w/w, 11% w/w to 50% w/w, 12% w/w to 50% w/w, 13% w/w to 50% w/w, 14% w/w to 50% w/w, and 15% w/w to 50% w/w.

In certain embodiments, the concentration of the cannabinoid in the topical composition may be within a range selected from the group consisting of:

1% w/w, 2% w/w to 40% w/w, 3% w/w to 40% w/w, 4% w/w to 40% w/w, 5% w/w to 40% w/w, 6% w/w to 40% w/w, 7% w/w to 40% w/w, 8% w/w to 40% w/w, 9% w/w to 40% w/w, 10% w/w to 40% w/w, 11% w/w to 40% w/w, 12% w/w to 40% w/w, 13% w/w to 40% w/w, 14% w/w to 40% w/w, and 15% w/w to 40% w/w.

In certain embodiments, the concentration of the cannabinoid in the topical composition may be within a range selected from the group consisting of:

1% w/w, 2% w/w to 30% w/w, 3% w/w to 30% w/w, 4% w/w to 30% w/w, 5% w/w to 30% w/w, 6% w/w to 30% w/w, 7% w/w to 30% w/w, 8% w/w to 30% w/w, 9% w/w to 30% w/w, 10% w/w to 30% w/w, 11% w/w to 30% w/w, 12% w/w to 30% w/w, 13% w/w to 30% w/w, 14% w/w to 30% w/w, and 15% w/w to 30% w/w.

In certain embodiments, the concentration of the cannabinoid in the topical composition may be within a range selected from the group consisting of:

1% w/w, 2% w/w to 20% w/w, 3% w/w to 20% w/w, 4% w/w to 20% w/w, 5% w/w to 20% w/w, 6% w/w to 20% w/w, 7% w/w to 20% w/w, 8% w/w to 20% w/w, 9% w/w to 20% w/w, 10% w/w to 20% w/w, 11% w/w to 20% w/w, 12% w/w to 20% w/w, 13% w/w to 20% w/w, 14% w/w to 20% w/w, and 15% w/w to 20% w/w.

### Other Agents

The cannabinoid could be incorporated into a composition with an additional active moiety that is capable of improving the appearance and/or hydration of the skin.

In addition, the composition of the present invention can be used in conjunction with other topically applied analgesic and/or systemically available agents for the treatment of acne.

Examples of such analgesic agents include, but are not limited to: morphine, cyclazocine, piperidine, piperazine, pyrrolidine, morphiceptin, meperidine, trifluadom, benzeneacetamine, diacylacetamide, benzomorphan, alkaloids, peptides, phenantrene and pharmaceutically acceptable salts, prodrugs or derivatives thereof. Specific examples of compounds contemplated by as suitable in the present invention include, but are not limited to morphine, heroin, hydromorphone, oxymorphone, levophanol, methadone, meperidine, fentanyl, codeine, hydrocodone, oxycodone, propoxyphene, buprenorphine, butorphanol, pentazocine and nalbuphine. As used in the context of opioid agents herein, “pharmaceutically acceptable salts, prodrugs and derivatives” refers to derivatives of the opioid analgesic compounds that are modified by, e.g., making acid or base salts thereof, or by modifying functional groups present on the compounds in such a way that the modifications are cleaved, either in routine manipulation or in vivo, to produce the analgesically active parent compound. Examples include but are not limited to mineral or organic salts of acidic residues such as amines, alkali or organic salts of acidic residues such as carboxylic acids, acetate, formate, sulfate, tartrate and benzoate derivatives, etc. Suitable opioid analgesic agents, including those specifically mentioned above, are also described in Goodman and Gilman, ibid, chapter 28, pp. 521-555.

Examples of systemically available agents which may be used in conjunction with the present compositions for the treatment of acne include, but are not limited to: retinoids such as tretinoin, isotretinoin, motretinide, adapalene, tazarotene, azelaic acid, and retinol; salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporin and cytokine synthesis inhibitors, tetracycline, minocycline, and doxycycline, or any combination thereof.

In addition, other active agents may be included in the composition of the present invention, e.g., topically-effective anaesthetics such as xylocaine, cocaine, lidocaine, benzocaine, etc., which may provide a more immediate, if less effective in the long run, level of pain relief until the analgesic agent becomes fully effective.

Still other agents can also be administered, preferably topically, to potentiate the effects of the topically-administered cannabidiol. For example, dextromethorphan, a non-addictive opioid compound, can be co-administered, preferably topically, although parenteral administration is also effective, to enhance the effectiveness of the topically administered agent. Without wishing to be bound by theory, it is believed that dextromethorphan has previously unappreciated analgesic properties in peripheral nerves. Suitable concentrations of dextromethorphan are routinely ascertainable by the skilled worker, and include the normal therapeutic amounts administered parenterally for conventional purposes, e.g., as a cough suppressant, or less, and routinely determinable amounts for topical administration; for example, 1 g of dextromethorphan can be added to a composition disclosed herein to provide additional treatment for acne.

In one embodiment, the pharmaceutical composition of the present invention further comprises one or more of the following agents for the treatment of acne: retinoids such as tretinoin, isotretinoin, motretinide, adapalene, tazarotene, azelaic acid, and retinol; salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporin and cytokine synthesis inhibitors, tetracycline, minocycline, and doxycycline, or any combination thereof.

### Acne Treatment and Therapy

In certain embodiments the topical application of cannabinoid, such as cannabidiol, by way of the compositions of the present invention is expected to reduce the incidence and/or severity of acne. Therapeutic effects of the present invention include, but are not limited to, reduction in redness, itch, pain or irritation, a reduction in pimples, papules, blisters or pustules, a reduction in infection, a reduction of swelling, cracking, weeping, crusting, and scaling and/or a general decrease in inflammation.

In certain embodiments, the topical application of cannabinoid, such as cannabidiol, by way of the compositions of the present invention is expected to improve the symptoms of acne.

The term “improve” is used to convey that the present invention changes either the appearance, form, characteristics and/or the physical attributes of the tissue to which it is being provided, applied or administered. The change in form may be demonstrated by any of the following alone or in combination: enhanced appearance of the skin; decreased inflammation of the skin, prevention of inflammation or blisters, decreased spread of blisters, decreased ulceration of the skin, decreased redness, reduction of scarring, reduction in lesions, healing of blisters, reduced skin thickening, closure of wounds and lesions, a reduction in symptoms including, but not limited to, pain, inflammation, itching, milia or other symptoms associated with inflammatory conditions or the like.

A primary advantage of the present invention is expected to be the improvement in the condition of the skin without the typical side effects of conventional therapies. The potential for the present invention is widespread, and the topical application of cannabinoids shows promise as an exciting new method of acne treatment.

It is expected that treatment of acne in accordance with embodiments of the present invention results in improved healing of the skin. For example, when used in the treatment of acne, swollen, cracked or scaled skin is which is treated is expected to heal more quickly and/or completely, compared to when left untreated.

When administered in accordance with the present invention, treatment is expected to result in one or more therapeutic effects. Therapeutic effects in the affected area include, but are not limited to, reduction in redness, itch, pain or irritation, the number and severity of the acne lesions, a reduction in infection, a reduction of swelling, cracking, weeping, crusting, and scaling and/or a general decrease in inflammation. One or more of these therapeutic effects are expected to be observed when treatment in accordance with the present invention is made to any of the suitable conditions.

The present invention further provides a method for treating or preventing acne in a patient in need of such treatment, the method comprising topically administering a prophylactically or therapeutically effective amount of a topical composition as described herein.

The present invention further provides the use of a cannabinoid and a siloxane for the manufacture of a topical composition, as described herein, for the prevention or treatment of acne in a patient in need thereof.

The present invention further provides the use of a topical composition, as described herein, for the prevention or treatment of acne.

In one aspect, the present invention is directed to methods of treating acne using topical cannabinoids, including cannabidiol. In accordance with certain embodiments, a topical composition of the invention containing cannabinoids such as cannabidiol, is preferably applied topically to an area which is affected by acne. Preferably, the application of cannabinoid in accordance with certain embodiments results in reduction in redness, itch, pain or irritation, a reduction in pimples, papules, blisters or pustules, a reduction in infection, less breakdown and loss of collagen and elastin in the skin, a reduction of swelling, cracking, weeping, crusting, and scaling and/or a general decrease in inflammation.

### Pharmaceutical Composition

Certain embodiments of the present invention comprise any topically acceptable non-transdermally effective carrier vehicle. Preferred topically acceptable vehicles include but are not limited to gels, ointments, and liquids. Administration of the preferred embodiment is performed in accordance with that mode which is most amenable to the topically acceptable form chosen. For example, gels, lotions, creams and ointments are preferably administered by spreading.

The dilution of the cannabinoid in the topical composition can be an important consideration. The cannabinoid concentration in the composition should be high enough that the patient does not need to wait an excessively long time for the composition to dry. On the other hand, the cannabinoid concentration should be dilute enough that a patient can achieve effective coverage of the affected area. Additionally, the composition could include a component which polymerizes in response to exposure to air or ultraviolet radiation.

The amount of composition to be applied will vary depending on the choice of siloxane, low molecular weight alcohol, fatty alcohol, and/or alkyl PEG/PPG ether as well. For example, when the cannabinoid, such as cannabidiol, is administered by spraying a solution of the drug, the total volume in a single dose may be as low as 0.1 ml. When the cannabinoid, such as cannabidiol, is administered in a gel or cream, the total volume may be as high as 3 ml. Conversely, if acne comprises scattered lesions, the volume applied to each lesion may be smaller. The carrier selected, and its manner of application, are preferably chosen in consideration of the needs of the patient and the preferences of the administering physician.

In one preferred embodiment, the composition comprises a gel which is preferably administered by spreading the gel onto the affected area. In other preferred embodiments, the composition comprises a liquid, which can be administered by spraying or otherwise applying the liquid onto the affected area.

The quantities of the applied cannabinoid, such as cannabidiol, described herein in the Examples are illustrative only and it is to be appreciated that lesser and greater quantities may be used, which can be routinely optimized by the skilled worker. In general, amounts therapeutically equivalent to 0.1 to 200 mg of cannabinoid, such as cannabidiol, applied to an area of 5-100 cm², are preferred. However, the quantity of cannabinoid used in the topical application of the present invention is typically a small fraction of the typical dosage used in other methods of treatment using these agents, e.g., epilepsy.

In accordance with certain embodiments, the composition is applied to the affected area regularly until relief is obtained. In one preferred embodiment, the composition is administered to the skin of the patient in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention.

In certain embodiments, the composition of the invention may be provided in a form selected from the group comprising, but not limited to a liquid or gel, a leave-on preparation, and a wash-off preparation.

In one embodiment, the composition comprises impurities, wherein the quantity of impurities as a percentage of the total weight of the composition is selected from the group consisting of: less than 20% impurities (by total weight of the composition); less than 15% impurities; less than 10% impurities; less than 8% impurities; less than 5% impurities; less than 4% impurities; less than 3% impurities; less than 2% impurities; less than 1% impurities: less than 0.5% impurities; less than 0.1% impurities. In one embodiment, the composition comprises microbial impurities or secondary metabolites, wherein the quantity of microbial impurities as a percentage of the total weight of the composition is selected from the group consisting of: less than 5%; less than 4%; less than 3%; less than 2%; less than 1% s; less than 0.5%; less than 0.1%; less than 0.01%; less than 0.001%. In one embodiment, the composition is sterile and stored in a sealed and sterile container. In one embodiment, the composition contains no detectable level of microbial contamination.

The foregoing embodiments are illustrative of applications in which methods of treating acne using a cannabinoid, such as cannabidiol, in accordance with the present invention can be employed. Those of ordinary skill in the art will readily understand that other manners of administration of cannabinoids to treat acne are suitable and are in accordance with the present invention as well.

### Definitions

The following definitions in this specification are intended to be interpreted in an illustrative, rather than limiting sense. Therefore, they are to be interpreted inclusively, and are not to be limited to the specific definition recited.

Antagonist: a compound that does not enhance or stimulate the functional properties of a receptor, yet block those actions by an agonist.

Bandage: a dressing used to cover an afflicted area.

Cannabinoid: as used herein, is meant to include compounds which interact with the cannabinoid receptor and various cannabinoid mimetics, such as certain tetrahydropyran analogs (e.g., Δ⁹-tetrahydrocannabinol, Δ⁸-tetrahydro-cannabinol, 6,6,9-trimethyl-3-pentyl-6H-dibenzo [b,d]pyran-1-ol, 3-(1,1-dimethylheptyl)-6, 6a, 7, 8, 10, 10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylheptyl,(+)-(3S,4S)-7-hydroxy-Δ6-tetrahydrocannabinol-1,1-dimethylheptyl, 11-hydroxy-Δ⁹-tetrahydrocannabinol, and 08-tetrahydrocannabinol-11-oic acid)); certain piperidine analogs (e.g., (−)-(6S,6aR,9R, 10aR)-5,6,6a,7,8,9,10,10a-octahydro-6-methyl-3-[(R)-1-methyl-4-phenylbutoxy]-1,9-phenanthridinediol-1-acetate)); certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone); and certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′,6′-hexahydrobiphenyl). Further examples of “cannabinoids” include those compounds described in the references cited below.

Cannabidiol: as used herein, is meant to refer to 2-[3-methyl-6-(1-methylethenyl)-2-cyclohexen-1-yl]-5-pentyl-1,3-benzenediol.

The synthesis of 2-[3-methyl-6-(1-methylethenyl)-2-cyclohexen-1-yl]-5-pentyl-1,3-benzenediol is described, for example, in Petilka et al., Helv. Chim. Acta, 52: 1102 (1969) and in Mechoulam et al., J. Am. Chem. Soc., 87:3273 (1965), which are hereby incorporated by reference

Central nervous system: the brain and spinal cord.

Dermal: relating to the dermis.

Dressing combine: designed to provide warmth and protection to absorb large quantities of fluid that may drain from an incision or wound; consists of a nonwoven fabric cover enclosing fibre with or without absorbent tissue.

Inflammation: an immune system-mediated process characterized by redness, heat, swelling, and pain at the local site.

Mammal: vertebrates with hair, three middle ear bones and mammary glands.

Mammals include humans.

Skin: the outer covering of an animal body. Mammalian skin comprises three layers: (i) an epidermis layer, which is predominantly composed of keratinocytes and a small number of melanocytes and Langerhans cells (antigen presenting cells); (ii) a dermis layer, which contains nerve endings, sweat glands and oil (sebaceous) glands, hair follicles, and blood vessels and which is primarily composed of fibroblasts; and (iii) a hypodermis layer of deeper subcutaneous fat and connective tissue. The epidermis itself is made up of two layers, the outer stratum corneum and the inner epidermal basal layer, sometimes referred to as the basement membrane. The purpose of the stratum corneum is to form a barrier to protect underlying tissue from infection, dehydration, chemicals and mechanical stress.

Therapeutically-effective amount: the amount necessary to bring about a therapeutic effect.

Transdermal: passing through the dermis.

### General

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The invention described herein may include one or more range of values (e.g. concentration). A range of values will be understood to include all values within the range, including the values defining the range, and values adjacent to the range which lead to the same or substantially the same outcome as the values immediately adjacent to that value which defines the boundary to the range.

The following Examples are to be construed as merely illustrative and not limitative of the remainder of the disclosure in any way whatsoever. These Examples are included solely for the purposes of exemplifying the present invention. They should not be understood as a restriction on the broad summary, disclosure or description of the invention as set out above. Without further elaboration, it is believed that one skilled in the art can, using the preceding description, utilize the present invention to its fullest extent. In the foregoing and in the following examples, all temperatures are set forth uncorrected in degrees Celsius; and, unless otherwise indicated, all parts and percentages are by weight.

### EXAMPLES

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### Example 1

### Example Techniques for Ascertaining Permeability of Compositions Containing Cannabidiol

The permeability of human skin has been studied for several decades. The skin consists of two major layers, the outer epidermis and the inner dermis. The stratum corneum (“SC”), the outermost 10-20 μm of the epidermis, is responsible for the skin's excellent diffusional resistance to the transdermal delivery of most drugs. Most of the skin's enzymatic activity lies in the basal cell layer of the viable epidermis. Fibrous collagen is the main structural component of the dermis. The skin vasculature is supported by this collagen and lies a few microns underneath the epidermis. Basically, it is here that permeation ends and systemic uptake begins. Many researchers have developed skin permeability relationships based on the physicochemical parameters (molecular weight, molecular volume, lipophilicity, hydrogen-bonding potentials, polarity, etc.) of skin penetrants. However, when dealing with transdermal administration of cannabinoids, these skin permeability relationships need to be modified to take into account the potential complications of extreme lipophilicity and concurrent metabolism of these drugs.

Selection and optimization of cannabinoids for delivery into the epidermis and dermis requires an understanding of their cutaneous metabolism. Furthermore, since skin metabolism of topical in vivo studies cannot easily be distinguished from blood, liver, or other tissue metabolism, cutaneous metabolism is better studied in vitro. However, the success of any such in vitro study depends heavily on finding ideal conditions to simulate in vivo conditions, especially in maintaining tissue viability. Thus, selection of an optimal receiver solution is critical to the success of any such in vitro studies.

A high-pressure liquid chromatography (HPLC) assay can be used for the analysis of cannabidiol in samples. An appropriate HPLC system may consist of a Waters 717 plus Autosampler, Waters 1525 Binary HPLC Pump and Waters 2487 Dual A Absorbance Detector with Waters Breeze software. A Brown-lee C-18 reversed-phase Spheri-5 μm column (220×4.6 mm) with a C-18 reversed phase 7 μm guard column (15×3.2 mm) may be used with the UV detector set at a wavelength of 215 nm. The mobile phase may comprise of acetonitrile: 25 mM phosphate buffer with 0.1% triethylamine pH 3.0 (80:20). An appropriate flow rate of the mobile phase would be 1.5 mL and 100 μL of the sample would be injected onto the column.

A PermeGear flow-through (In-Line, Riegelsville, Pa.) diffusion cell system is appropriate for the skin permeation studies. Trans-epidermal water loss can be measured (Evaporimeter EPI™, ServoMed, Sweden) after securing the skin in the cells. Pieces of skin with readings below 10 g/m2/h would be used for the diffusion studies. The skin surface in the diffusion cells would be maintained at 32° C. with a circulating water bath. An appropriate receiver solution would be HEPES-buffered Hanks' balanced salts with gentamicin (to inhibit microbial growth) containing 40% polyethylene glycol 400 (pH 7.4), and the flow rate was adjusted to 1.1 mL/h. An excess quantity of CBD would be added to the donor vehicle (propylene glycol: Hanks' buffer (80:20)) solution with and without permeation enhancers at 6% v/v, sonicated for 10 min, and then applied onto the skin. Excess quantity of the drug would be used in the donor compartment throughout the diffusion experiment in order to maintain maximum and constant chemical potential of the drug in the donor vehicle. Each cell would appropriately be charged with 0.25 mL of the respective drug solution. Samples would appropriately be collected in 6 h increments for 48 h. All the samples would appropriately be stored at 4° C. until HPLC analysis.

Drug disposition in the skin samples would be measured at the completion of the 48 h experiment. The skin tissue would be rinsed with nanopure water and blotted with a paper towel. To remove the drug formulation adhering to the surface, the skin would be tape stripped twice using book tape (Scotch®, 3M, St. Paul, Minn.). The skin in contact with the drug would be excised, minced with a scalpel and placed in a pre-weighed vial. Drug would be extracted from the skin by equilibrating with 10 mL of ACN in a shaking water bath overnight at room temperature. Samples would be analyzed by HPLC to determine CBD content in micromoles (μm) of drug per gram of wet tissue weight. Statistical analysis of the in vitro human skin permeation data could be performed using SigmaStat 2.03. A one-way ANOVA with Tukey post-hoc analysis could be used to test the statistical differences among the different treatments.

The results of such a study are expected to indicate that cannabidiol can be delivered via the topical route using compositions according to the present invention, and that siloxanes, low molecular weight alcohols, fatty alcohols and/or alkyl PEG/PPG ether increase the amounts of cannabidiol delivered into human skin.

### Example 2

### Objective:

To prepare formulations of cannabidiol with a siloxane, together with other excipients.

Methods and Results from the Initial Solubility Studies:

First, the solubility of cannabidiol (CBD) was assessed. The powder looked granulated under the microscope. The solubility (weight to weight) of CBD was under about 3-4% in hexamethyldisiloxane (HDS) and mineral oil. The solubility in propylene glycol (PG) and ethanol was about 6-7%, although the reported solubility in ethanol is 3.5%. The solubility in oleyl alcohol (OA) was greater than 8% (did not go higher in studies) and the solubility in isopropyl alcohol (IPA) was greater than 14%. The conclusions from the solubility studies were that OA and IPA were very good solvents and it was surprising that IPA was so much better than ethanol. The solubility in HDS and mineral oil was low, so a completely nonpolar solvent does not work well to dissolve high levels of CBD, but the addition of an OH group present in a fatty alcohol really increased the CBD solubility.

Second, the CBD was dissolved at a moderate concentration in a highly volatile solvent with some nonvolatile solvents that would keep CBD in solution (non-crystalline), i.e., prevent crystallization at high concentrations (of the order of 40-50%).

### Formulations:

The following formulations were prepared:
a) Form I: 5% CBD/10% OA/10% PG/10% HDS/65% IPA (some HDS was added because it has little odour, is very volatile, and reduced irritation). The residual concentration of CBD in the PG/OA would be 20%, which appeared a suitable good target. A drop of the formulation was placed on a microscope slide and there was no CBD crystallization post evaporation of highly volatile solvents. The residue remained crystal free after an hour, so more CBD was added to make a 14% CBD/9% OA/9% PG/9% HDS/59% IPA solution. The residual concentration of CBD was then 44% CBD, still no CBD crystals after evaporation. Even overnight, no crystals were observed.
b) Form II: 14% CBD/4.5% OA/13.5% PG/4.5% HDS/63.5% IPA. This solution also did not form crystals in one hour or overnight.
c) Form III: 8% CBD in IPA. No crystals after an hour but overnight there were needle-like crystals that looked clear, not yellowish, under the microscope. The film of just liquid CBD in the microscope slide and on skin was of high friction, and probably would not be so acceptable to patients. A 10% solution in IPA applied to 1 cm² would give about a 10 micron thick layer (10 mg), about the thickness of stratum corneum. Made up 15% CBD in IPA and 15% CBD in 50/50 IPA/HDS with no crystals immediately.
d) Both Form I and Form II were thickened with 1% Klucel MF. Both took several minutes to become less tacky and neither of them formed crystals even after two days (samples on microscope slides). Form III was also gelled and was tacky.
e) Form IV: 3% CBD/9% PMS/88% HDS This solution was placed on a microscope slide and as the HDS evaporated the PMS was left with tiny spheres of CBD dispersed in the PMS. It was not tacky on the skin. No crystals appeared that day but overnight needle crystals appeared. Residual is 25% CBD.
f) Form V: OA was added to form IV to prevent overnight crystallization. It was 7.6% CBD/8% OA/8% PMS/76.4% HDS with a residual CBD of 32%. There were no crystals overnight. Added further CBD and PMS to make 10% CBD/7.7% OA/8.7% PMS/73.6HDS with a residual of 38% CBD and with similar feel and no crystals.
g) Form VI: 14% CBD/6% OA/6% PG/10% HDS/64% IPA with a residual of 54% CBD. This formulation had crystals after 48 hours. Added Klucel and only a few crystals after 48 hours. It was less tacky than the other two gels with higher OA and PG.
h) Form VII: 15% CBD/10% argan/10% HDS/65% IPA with residual of 60% CBD. A few crystals were observed after 2-3 hours. After adding Klucel, the gel had a better feel than the ones with PG and OA.
i) Form VIII: 15% CBD/5% PMS/10% OA/70% HDS. Good feel and no crystals.
j) Form IX: 10% CBD/7% argan/7% ISA/9% PMS/67% HDS. No crystals.
k) Form X: 15% CBD/13% HSA/7% PMS/66% HDS with a residual of 43% CBD. No crystals.
l) Form XI: 15% CBD/12.5% H/6% PMS/66.5% HDS with a residual CBD of 45%. No crystals, just droplets in PMS.
m) Form XII: 15% CBD/12.5% ODDA/6% PMS/66.5% HDS with a residual CBD of 45%. No crystals, just droplets in PMS.
n) Form XIII: 15% CBD/10% H/40% IPA/35% HDS with a residual CBD of 60%. No crystals. Reason for reducing IPA was to reduce potential for stinging, odour, and cooling.
o) Form XIX: 15% CBD/10% ODDA/40% IPA/35% HDS with a residual CBD of 60%. No crystals.
p) Added Klucel to Form XIII and XIX. They were not as viscous, since the HDS level was high, but they felt very good on the skin and not so tacky.
q) Form XX: 7.2% CBD/6.3% PMS/1.4% MO/1.8% IPA/83.3% HDS. No crystal of CBD and great feel with a residual CBD of 48%.
r) Form XXI: A higher CBD concentration was made: 20% CBD/10% ODDA/70% IPA with a residual CBD of 67% and no crystals.
s) Form XXII: 9.5 CBD/4.8% ODDA/57.1% EtOH/28.6% HDS with no crystals and a residual CBD of 66%.
t) Form XXIII: 10% CBD/12.5% PMS/4.5% IPA/72% HDS with good feel and no crystals with a residual CDB of 42%. Added about 4% petrolatum and had a hazy solution (from petrolatum) with no crystals.

### Example 3

### Objective:

To prepare further formulations of cannabidiol with a siloxane, together with other excipients.

### Methods:

CBD2 is an off-white powder of crystals that produced clear solutions in marked contrast to CBD1 solutions that were colored by the end of the day. None of the CBD2 solutions were colored at the end of day 1 and looked clear. The CBD2 material dissolved like the CBD1 therefore the CBD2 is CBD without the discoloration properties of CBD1.

### Formulations

Formulation A (Form A)

### 5% CBD/2.5% HDA/1% PMS/91.5% HDS

A repeat of the acne “spray on” formulation A-7 was conducted and it behaved the same except it did not have any discoloration and was clear. It showed no signs of discoloration by the end of the day.

Tests Performed:

a) A drop on a microscope slide covered about 1 cm² and no crystals appeared until later in the day (about 4 hours later) when it was rubbed vigorously with a finger, which resulted in crystal growth.
b) Drops of Form A were placed on the skin and spread around with a finger. It dried quickly and was smooth and transparent on the skin. These results were consistent with the behavior of A-7 with CBD1.
c) Drops of Form A were spread and rubbed lightly onto the back of the hand, and after 5 minutes a microscope slide was pressed hard against the skin and some material was transferred to the slide. Under the microscope slide there were some CBD crystals. It is a transparent film. It appears that if the film is not mechanically disturbed, crystals do not form, but with rubbing, some crystals are formed.
d) Added about 100 mg of PMS to Form A to make it about 3% PMS vs. 1%. This appeared to reduce the crystallization using the skin blot technique, but this was only a qualitative observation.

Formulation B

### 5% CBD/1.7% HDA/1.2% PMS/92.1% HDS

This formulation was made to determine if HDA could be reduced slightly. It appeared in all the tests to be similar to Form A.

Formulation C

### 5.25% CBD/1.15% PMS/1.22% IPA/92.38% HDS

The objective of this formulation was to determine whether HDA could be replaced by IPA.

Tests Performed:

A drop of Form C was placed on a microscope slide and it spread out to make clear film, which quickly became a white film. Under the microscope there were tiny crystals stuck together by the PMS. When placed on the skin, it turned chalky white as well. The inventors tried adding additional PMS up to about 5% but that did not end the chalkiness, although it slowed the rate down.

### Example 4

### Objective:

To determine if arlamol E (AE) or isopropyl myristate (IPM) could replace hexyldecyl alcohol (HDA), since they are both used in pharmaceutical topical products for the acne formulation 5% CBD/2.5% HDA/1% PMS/91.5% HDS.

### Summary:

AE, which was initially avoided due to an intense purple color using CBD 1, was found to be the best replacement and even superior to HAD. It did have a slight purple color when CBD was dissolved in pure AE at the 10% level but not in the formulations using AE.

### Results:

Solubility studies: CBD dissolved at the 10% level in AE and barely 9.5% in IPM. Further exploration was not conducted due to the small amount of drug API available for non-GMP work. CBD is soluble greater than 10% but probably not in excess of 20%, as the time to dissolve additional CBD was taking considerably longer.

Five grams each of 5% CBD/2.5% AE/1% PMS/91.5% HDS and 5% CBD/1% AE/1% PMS/93% HDS formulations were made and investigated for crystal formation after evaporation of HDS. The purpose of reducing AE was to evaluate if we could further reduce AE from the 2.5% level, which did not produce crystals on a microscope slide plus or minus rubbing or no the skin as seen from an imprint on a slide pressed hard on the skin after rubbing in the formulation. After rubbing the 1% AE formulation deposited on a slide, crystal began to form rapidly. After rubbing the 2.5% AE formulation one could observe many very tiny (smaller than a period at 100×) droplets (no crystals). Since the formulation minus CBD did not produce these droplets, it was hypothesized that the droplets are “supersaturated CBD in AE”. Without rubbing the droplets are not created and the formulation looks like a clear film.

Five grams of a 5% CBD/2.5% IPM/1% PMS/1% IPA/90.5% HDS formulation were made. IPA was added to completely dissolve the CBD. This formulation produced crystal growth rapidly as the formulation was rubbed while drying on a slide as contrasted with the AE formulation.

Five grams of a 10% CBD/4% AE/1% PMS/1% IPA/84% HDS formulation were made (IPA added to completely dissolve the CBD). This formulation did not produce crystals of CBD upon evaporation and rubbing on a slide although it had a reduced ration of CBD:AE. The residual solution of CBD in AE would be 71%.

Formulation Recommendations are:

5% CBD/2% AE/1% PMS/92% HDS

10% CBD/4% AE/1% PMS/1% IPA/84% HDS

### Example 5

### Objective:

To test several more formulations at the 5%, 10%, and 15% CBD concentrations.

### Methods:

The acne formulations were alcohol (isopropyl alcohol [IPA]) based to allow for thickening with Klucel and silioxane (hexylmethyldisiloxane [HDS]) based for spray on formulations. The psoriasis formulations were siloxane based and thickened with polymethylsiloxane 10⁶ cSt (PMS). All the formulations would be suitable for human studies, and under microscope evaluation post evaporation all formulations did not crystalize CBD. The residual solubilizer was 2-hexyldecyl alcohol (HDA) and residual concentrations were 60% to 67%.

### Formulations

### Acne “Gels”

A-1: 5% CBD/2.5% HDA/50% IPA/41% HDS/1% KlucelMF

At 1% Klucel this gel and all the 1% Gels were basically thickened such that they could be applied from a dropper container to spread on the skin. Additional Klucel was added to this formulation, which became much stiffer.

A-2: 5% CBD/3.33% HDA/50% IPA/40.67% HDS/1% KlucelMF

A-3: 5% CBD/3.33% HDA/75% IPA/15.67% HDS/1% KlucelMF

A-4: 10% CBD/6.67% HDA/75% IPA/7.33% HDS/1% KlucelMF

A-5: 15% CBD/10% HDA/70% IPA/4% HDS/1% KlucelMF

A-6: 15% CBD/7.5% HDA/70% IPA/6% HDS/1.5% KlucelMF

This formulation had 0.5% more Klucel and was more viscous.

Acne “Spray On”

A-7: 5% CBD/2.5% HDA/1% PMS/91.5% HDS

A-8: 10% CBD/5% HDA/1% PMS/84% HDS

A-9: 15% CBD/7.5% HDA/1% PMS/1% IPA/1% D5/74.5% HDS

1% IPA was added because the CBD was not quite soluble at 15% without IPA.

Observations of the formulations indicated that the formulations (which were not light protected) with alcohol tended to be darker with time than those without alcohol.

Psoriasis Formulations (Similar to the Acne Spray but with More PMS)

P-1: 5% CBD/2.5% HDA/5% PMS/87.5% HDS

P-2: 10% CBD/6.67% HDA/5% PMS/78.33% HDS

P-3: 15% CBD/7.5% HDA/5% PMS/1% IPA/71.5% HDS

P-4: 15% CBD/7.5% HDA/10% PMS/1% IPA/66.5% HDS

As for the acne spray on formulations 1% IPA was employed for the 15% CBD formulations.

### Example 6

An Open-Label Phase 1a Study to Evaluate the Safety and Tolerability of BTX 1503 Solution in Healthy Volunteers. The objectives of this study were to determine the safety, tolerability, and pharmacokinetics (PK) of a single dose and 14 days of treatment with BTX 1503 in healthy volunteers.

### Methodology:

Test Product, Dose and Mode of Administration, Batch Number:
Test Product: BTX 1503-5% (w/w) Solution. Contains the active pharmaceutical ingredient, cannabidiol (CBD; 2-[(1R,6R)-6-isopropenyl-3-methylcyclohex-2-en-1-yl]-5-pentylbenzene-1,3-diol).
Administration: One or 3 mL of the study drug was applied topically to the face once (QD) or twice (BID) daily (at about the same time each day) using an applicator swab.
Batch Number: PPP.17.566
Single-dose on Day 1, then multiple dosing starting on Day 8 for 14 days to Day 21

TABLE 2
Composition of 5% BTX 1503

| Ingredients | 5% Solution (% w/w) |
|---|---|
| Hexamethyldisiloxane (HDS) | 92.0 |
| Polydimethylsiloxane | 1.0 |
| Polypropylene Glycol-15 (PPG-15) Stearyl Ether | 2.0 |
| Cannabidiol (CBD) | 5.0 |

Number of Participants: 20 participants (5 participants in each cohort). This study included male and female participants who were between 18 and 65 years of age (inclusive). Participants were in good general health without clinically significant disease.

Eligible healthy volunteers were assigned in sequential cohorts (Cohorts 1, 2, 3, and 4) and received either a single (QD) application of study drug on Day 1 or BID (12 hours apart).
Cohort 1: 37.5 mg CBD/day (0.066 mg/cm²/day) applied as 1 mL of BTX 1503 5% (w/w) QD
Cohort 2: 75 mg CBD/day (0.133 mg/cm²/day) applied as 1 mL of BTX 1503 5% (w/w) BID
Cohort 3: 112.5 mg CBD/day (0.199 mg/cm²/day) applied as 3 mL of BTX 1503 5% (w/w) QD
Cohort 4: 225 mg CBD/day (0.398 mg/cm²/day) applied as 3 mL of BTX 1503 5% (w/w) BID

Participants were confined to the clinical site for the first 24 hours after the first dose. Blood draws for CBC and chemistry, and urine samples for urinalysis, were obtained prior to and at 12 hours after the first dose. Blood samples for PK analysis were obtained at pre-dose (within 15 minutes before dosing), 30, 60 and 90 minutes and 2, 2.5, 3, 4, 6, 8 and 12 hours, and 24 hours after the first single dose. For participants receiving BID dosing, samples were also taken at 30, 60 and 90 minutes and 2, 2.5, 3, 4, 6, and 8 hours after the second dose on Day 1.

Safety was monitored throughout the 24 hours after the first study drug application. Cutaneous tolerability was monitored throughout the 24-hour in-clinic period and reported at 1, 2, 4, 8, 12 and 24 hours after the first dose. After the single dose of study drug, participants had a washout period and returned to the clinic on Day 8 at which time safety assessments, cutaneous tolerability, and blood draws for study drug levels were obtained.

At the Day 8 Visit, participants received their first QD or BID study drug application at the clinical site for the multiple-dose (14-day) phase. Participants received dosing through Day 21. All participants returned to the clinical site daily. For participants receiving QD dosing, dosing occurred each day at the clinical site at approximately the same time (±1 hour) in the morning. Participants receiving BID dosing were instructed in how to apply study drug when not at the clinical site. For participants receiving BID dosing, the second application was self-administered by the participant 12 hours (±1 hour) later. For participants that self-administered the study drug, a diary was maintained documenting compliance. The clinical site or participants applied the total amount of study drug per application evenly, as best as possible, to cover the entire face.

At the Day 15 Visit, in addition to daily study drug application and cutaneous tolerability assessments, safety assessments and blood draws for study drug trough levels were obtained.

At the Day 21 Visit, the clinical site applied the final dose(s) and PK samples through 24 hours were obtained. Participants remained in the clinic for 24 hours. On Day 21, safety assessments were performed. Cutaneous tolerability assessments were conducted at 1, 12, and 24 hours (Day 22) after application of the final dose. Participants returned 48 hours (Day 23) after the final dose for a final blood draw for blood levels and a final cutaneous tolerability assessment. Urine drug tests were conducted on Day 1 and Day 21 at 12 and 24 hours after application to assess for the presence of THC. Adverse events and concomitant medications were monitored throughout the study.

### Criteria for Evaluation

Safety and tolerability were the primary outcome measures. For each treatment cohort of escalating dose, participants were monitored for adverse events (AEs) and local cutaneous tolerability. If a cohort met the definition of the maximum tolerated dose (MTD), the subsequent cohort was not to begin.

Safety was assessed through collection of vital signs (temperature, blood pressure and pulse), AEs, and laboratory findings (CBC, chemistry, and urinalysis). Vital signs were obtained prior to and 2, 4, 8, and 24 hours after the first study drug application on Day 1 and prior to study drug application on Day 8, Day 15, and Day 21. Adverse events were monitored from time of consent through the end of study. Complete blood count (CBC), chemistry, and urinalysis were conducted at Baseline prior to and 12 hours after the first dose and prior to dosing on Day 8, Day 15, and Day 21. Urine was collected prior to and at 12 hours and 24 hours after application of study drug on Day 1 and on Day 21 to evaluate for drugs of abuse, specifically delta-9-tetrahydrocannabinol (THC).

### Treatments Administered

Botanix Pharmaceuticals' BTX 1503 contains the active pharmaceutical ingredient, cannabidiol (CBD). The drug product is a clear liquid solution with a 5% (w/w) concentration of CBD.

Each milliliter of the 5% BTX 1503 solution contained 37.5 mg of CBD. Participants in Cohort 1 applied a maximum of 37.5 mg of CBD to the face daily, participants in Cohort 2 applied a maximum of 75 mg of CBD to the face daily, Cohort 3 applied a maximum of 112.5 mg of CBD to the face daily, and Cohort 4 applied a maximum of 225 mg to the face daily. Study drug was applied to the face using a supplied dry swab.

The starting dose in Cohort 1 was a single dose of 1 mL of study drug applied to the entire face. Following a washout period, with no identification of a maximum tolerated dose (MTD), participants in Cohort 1 began the 14-day multiple-dose phase at 1 mL of study drug applied QD.

With no identification of a MTD after the single dose and washout period in Cohort 1, Cohort 2 will begin with a dose of 1 mL applied twice on Day 1, 12 hours (±1 hour) apart (BID). Following a washout period, with no identification of a MTD, participants in Cohort 2 began the 14-day multiple-dose phase at 1 mL of study drug applied BID.

With no identification of a MTD after the Day 1 dosing and washout period in Cohort 2, Cohort 3 will begin with a single dose of 3 mL on Day 1 (QD). Following a washout period, with no identification of a MTD, participants in Cohort 3 began the 14-day multiple-dose phase at 3 mL of study drug applied QD.

With no identification of a MTD after the single dose on Day 1 and washout period in Cohort 3, Cohort 4 will begin with a dose of 3 mL applied twice on Day 1, 12 hours (±1 hour) apart (BID). Following a washout period, with no identification of a MTD, participants in Cohort 4 began the 14-day multiple-dose phase at 3 mL of study drug applied BID.

### Safety Evaluation

Safety was primarily evaluated through a review of adverse events and the investigator's assessments of cutaneous tolerability (erythema, scaling, dryness, burning/stinging, and irritant/allergic contact dermatitis). Signs and symptoms of cutaneous tolerability were graded using the following scale: 0, None; 1, Slight; 2, Moderate; 3, Intense. Cutaneous tolerability was assessed at each visit.

Complete blood count (CBC), chemistry, and urinalysis were conducted at Baseline prior to and 12 hours after the first dose and prior to dosing on Days 8, 15, and 21. The following were assessed:
CBC: White blood cell (WBC) count (with automated differential for absolute neutrophils, lymphocytes, monocytes, eosinophils, and basophils), red blood cell (RBC) count, haemoglobin, hematocrit, mean corpuscular volume (MCV), mean corpuscular haemoglobin (MCH), mean corpuscular haemoglobin concentration (MCHC), and platelet count
Chemistry: Glucose, albumin, total protein, calcium, sodium, potassium, chloride, CO2 (bicarbonate), urea, creatinine, alkaline phosphatase, alanine amino transferase (ALT), aspartate amine transferase (AST), and total bilirubin
Urinalysis: Color, clarity, specific gravity, pH, protein, glucose, leukocyte esterase using a dipstick. If the results are abnormal, a sample will be sent to the central lab for full urinalysis including microscopic analysis for red blood cells, white blood cells, squamous epithelial cells, and culture.

Urine drug testing, including for the presence of THC, were measured using a urine drug screening kit at 12 and 24 hours after application on study Day 1 and Day 21.

### Pharmacokinetics

Blood samples were taken for PK assessments on Day 1 (Baseline) at pre-dose (within 15 minutes before dosing), 30, 60 and 90 minutes and 2, 2.5, 3, 4, 6, 8 and 12 hours, and 24 hours after the first single dose. For participants receiving BID dosing, samples were also taken at 30, 60 and 90 minutes and 2, 2.5, 3, 4, 6, and 8 hours after the second dose on Day 1.

During the multiple-dose (14-day) phase, trough levels were obtained before the morning application on Day 15. On Day 21, blood samples were taken for PK assessments at pre-dose (within 15 minutes before dosing), 30, 60 and 90 minutes and 2, 2.5, 3, 4, 6, 8 and 12 hours, 24 hours and 48 hours after the morning dose. For participants receiving BID dosing, samples were also taken at 30, 60 and 90 minutes and 2, 2.5, 3, 4, 6, and 8 hours after the evening dose on Day 21.

### Statistical Methods:

All statistical processing was performed using SAS® 9.4.

The PK parameters for BTX 1503 Solution were calculated with the PK software Phoenix™ WinNonlin® Version 7.0 and analysed at Day 1 and at Day 21 (Cmax, tmax, AUC0-t, t½, and AUCO-∞) using model-independent methods. Trough levels on Day 15 were summarised.

Demographics were summarised by age, gender, race, ethnicity height and weight. For continuous variables, the mean, standard deviation (SD), median, and range were presented. Categorical variables were summarised by proportions.

### Drug Levels:

Blood levels of study drug were summarised by nominal time point. The mean, SD, median and range were presented.

### Sample Size:

The selected sample size was based on having appropriate sensitivity to observe a safety signal and to assess PK. Twenty participants (5 in each cohort) receiving active BTX 1503 was considered adequate to detect if there are any cutaneous or systemic safety or tolerability concerns.

### Demographics and Baseline Characteristics:

Participants ranged in age from 19 to 57 years, with a mean (standard deviation [SD]) age of 35.1 (12.27) years. The participants were approximately balanced by sex (55.0% male and 45.0% female), and were predominantly not Hispanic or Latino (95.0%) and White (90.0%). Cohort 1 and Cohort 3 were balanced by gender. Cohort 2 was predominantly female (80%) and Cohort 4 exclusively male.

Participants administered a similar mean number of study drug applications, 15 for the QD cohorts (Cohort 1 and Cohort 3) and approximately 30 for the BID Cohorts (Cohort 2 and Cohort 4). Only one participant in Cohort 4 missed dosing (morning and evening) on Day 18. All other participants received all their scheduled dosing.

TABLE 3
Participant Demographics

| | | Cohort 1 | | Cohort 2 | | Cohort 3 | | Cohort 4 | | All | |
|---|---|---|---|---|---|---|---|---|---|---|---|
| | | 1 mL QD | | 1 mL BID | | 3 mL QD | | 3 mL BID | | Participants | |
| Age (years) | n | 5 | | 5 | | 5 | | 5 | | 20 | |
| at Screening | Mean | 38.4 | | 41.2 | | 31.0 | | 29.6 | | 35.1 | |
| | SD | 15.53 | | 9.81 | | 14.37 | | 7.20 | | 12.27 | |
| | Median | 44.0 | | 47.0 | | 26.0 | | 28.0 | | 34.0 | |
| | Minimum | 19 | | 27 | | 20 | | 21 | | 19 | |
| | Max | 57 | | 50 | | 55 | | 37 | | 57 | |
| Gender n (%) | Male | 2 | (40.0%) | 1 | (20.0%) | 3 | (60.0%) | 5 | (100.0%) | 11 | (55.0%) |
| | Female | 3 | (60.0%) | 4 | (80.0%) | 2 | (40.0%) | 0 | (0.0%) | 9 | (45.0%) |
| Ethnicity n (%) | Hispanic or Latino | 1 | (20.0%) | 0 | (0.0%) | 0 | (0.0%) | 0 | (0.0%) | 1 | (5.0%) |
| | Not Hispanic or Latino | 4 | (80.0%) | 5 | (100.0%) | 5 | (100.0%) | 5 | (100.0%) | 19 | (95.0%) |
| Race n (%) | White | 4 | (80.0%) | 5 | (100.0%) | 5 | (100.0%) | 4 | (80.0%) | 18 | (90.0%) |
| | Aboriginal | 0 | (0.0%) | 0 | (0.0%) | 0 | (0.0%) | 0 | (0.0%) | 0 | (0.0%) |
| | Asian | 0 | (0.0%) | 0 | (0.0%) | 0 | (0.0%) | 0 | (0.0%) | 0 | (0.0%) |
| | Torres Strait Islander | 0 | (0.0%) | 0 | (0.0%) | 0 | (0.0%) | 0 | (0.0%) | 0 | (0.0%) |
| | Black or African American | 0 | (0.0%) | 0 | (0.0%) | 0 | (0.0%) | 1 | (20.0%) | 1 | (5.0%) |
| | Other | 1 | (20.0%) | 0 | (0.0%) | 0 | (0.0%) | 0 | (0.0%) | 1 | (5.0%) |

SD: Standard Deviation.

TABLE 4
Baseline Characteristics

| | | Cohort 1 | | Cohort 2 | | Cohort 3 | | Cohort 4 | | All | |
|---|---|---|---|---|---|---|---|---|---|---|---|
| | | 1 mL QD | | 1 mL BID | | 3 mL QD | | 3 mL BID | | Participants | |
| | | (n = 5) | | (n = 5) | | (n = 5) | | (n = 5) | | (n = 5) | |
| Height (cm) | n | 5 | | 5 | | 5 | | 5 | | 20 | |
| at Screening | Mean | 172.86 | | 169.58 | | 176.26 | | 177.54 | | 174.06 | |
| | SD | 6.240 | | 5.934 | | 11.039 | | 7.764 | | 8.005 | |
| | Median | 176.40 | | 166.50 | | 178.40 | | 181.00 | | 176.40 | |
| | Minimum | 162.5 | | 163.5 | | 160.0 | | 164.2 | | 160.0 | |
| | Max | 177.5 | | 177.0 | | 186.0 | | 183.5 | | 186.0 | |
| Weight (kg) | n | 5 | | 5 | | 5 | | 5 | | 20 | |
| at Screening | Mean | 66.28 | | 79.42 | | 77.92 | | 91.92 | | 78.89 | |
| | SD | 6.730 | | 15.146 | | 21.284 | | 21.933 | | 18.475 | |
| | Median | 66.20 | | 80.00 | | 75.40 | | 92.10 | | 74.40 | |
| | Minimum | 58.8 | | 59.1 | | 54.2 | | 56.9 | | 54.2 | |
| | Max | 73.4 | | 97.8 | | 112.1 | | 115.4 | | 115.4 | |
| Alcohol Use | Non-drinker | 2 | (40.0%) | 0 | (0.0%) | 0 | (0.0%) | 0 | (0.0%) | 2 | (10.0%) |
| n (%) | Drinker | 3 | (60.0%) | 5 | (100.0%) | 5 | (100.0%) | 5 | (100.0%) | 18 | (90.0%) |
| Smoking | Never Smoked | 4 | (80.0%) | 4 | (80.0%) | 4 | (80.0%) | 4 | (80.0%) | 16 | (80.0%) |
| Status n (%) | Former Smoker | 1 | (20.0%) | 0 | (0.0%) | 1 | (20.0%) | 1 | (20.0%) | 3 | (15.0%) |
| | Current Smoker | 0 | (0.0%) | 1 | (20.0%) | 0 | (0.0%) | 0 | (0.0%) | 1 | (5.0%) |

SD: Standard Deviation.

Safety Results:

Of adverse events, facial dryness was most frequently reported. It is important to note that facial moisturisers were prohibited during the study.

There were no clinically relevant changes from baseline observed in safety laboratory assessments (CBC, chemistry, and urinalysis), or in vital signs (blood pressure, temperature and pulse). Participants were also tested for the presence of THC using a urine drug test. THC was not observed.

### Pharmacokinetics:

The pharmacokinetics of CBD after a single-dose and after 14 days of either QD or BID dosing with BTX 1503 5% Solution demonstrated that systemic absorption was low and dose dependent, although not dose proportional. CBD levels were first observed between 2 and 3 hours after initial dosing. Tmax occurred at 18 hours (Cohort 1) and 10 hours (Cohort 3) after QD dosing and at 19 to 20 hours (after the first dose was administered) for BID dosing. Levels of CBD were below the limits of quantitation (BLOQ; <0.2 ng/mL) for all participants in Cohorts 3 and 4 by study Day 8, seven days after the initial single dose. By Day 21, CBD levels appeared to be at steady state. The maximum mean AUC(0-48) and Cmax at the end of the multiple-dose phase were 63.87 (±30.483) h*ng/mL and 2.17 (±1.209) ng/mL, respectively. The Accumulation Ratios for AUC(0-24) were consistent for all cohorts ranging from 1.92 to 2.74 indicating that there was limited accumulation.

TABLE 5
Mean Pharmacokinetic Values for Clinical Study No. BTX.2017.001

| Cohort 1 | | Cohort 2 | | Cohort 3 | | Cohort 4 | |
|---|---|---|---|---|---|---|---|
| 37.5 mg/day | | 75 mg/day | | 112.5 mg/day | | 225 mg/day | |
| n | Mean (±SD) | n | Mean (±SD) | n | Mean (±SD) | n | Mean (±SD) |

Day 1

| | | | | | | | | | | | | |
|---|---|---|---|---|---|---|---|---|---|---|---|---|
| AUC₍₀₋ₜ₎ (h*ng/mL) | 5 | 5.72 | (3.748) | 5 | 7.49 | (2.120) | 5 | 9.54 | (3.419) | 5 | 11.69 | (2.986) |
| AUC₍₀₋₂₄₎ (h*ng/mL) | 4 | 7.13 | (2.267) | 5 | 7.44 | (2.087) | 4 | 10.72 | (2.499) | 5 | 11.69 | (2.989) |
| Cₘₐₓ (ng/mL) | 5 | 0.35 | (0.202) | 5 | 0.57 | (0.145) | 5 | 0.43 | (0.187) | 5 | 0.89 | (0.246) |
| Tₘₐₓ (h) | 4 | 18.03 | (6.938) | 5 | 19.08 | (1.060) | 5 | 10.41 | (2.197) | 5 | 19.68 | (1.170) |

| t_{1/2} (h) | 5 | — | 5 | — | | 5 | — | | 5 | — | |
|---|---|---|---|---|---|---|---|---|---|---|---|
| Day 15 (ng/mL) | 5 | — | 5 | 0.78 | (0.599) | 5 | 0.57 | (0.232) | 5 | 2.11 | (2.182) |

Day 21

| | | | | | | | | | | | | |
|---|---|---|---|---|---|---|---|---|---|---|---|---|
| AUC₍₀₋₂₄₎ (h*ng/mL) | 5 | 13.90 | (4.769) | 5 | 18.63 | (6.538) | 5 | 19.93 | (3.434) | 5 | 31.85 | (12.790) |
| AUC₍₀₋₄₈₎ (h*ng/mL) | 4 | 21.13 | (2.842) | 4 | 32.36 | (7.897) | 5 | 30.43 | (4.549) | 5 | 63.87 | (30.483) |
| Cₘₐₓ (ng/mL) | 5 | 0.83 | (0.302) | 5 | 1.17 | (0.565) | 5 | 1.26 | (0.402) | 5 | 2.17 | (1.209) |
| Tₘₐₓ (h) | 5 | 7.90 | (4.633) | 5 | 12.94 | (7.407) | 5 | 8.00 | (2.450) | 5 | 5.46 | (10.370) |
| t_{1/2} (h) | 3 | 66.79 | (67.914) | 4 | 22.76 | (8.669) | 4 | 28.27 | (8.063) | 4 | 38.08 | (26.957) |
| AR AUC₍₀₋ₜ₎ | 4 | 3.74 | (0.927) | 5 | 4.09 | (2.571) | 5 | 3.80 | (2.220) | 5 | 5.45 | (1.984) |
| AR AUC₍₀₋₂₄₎ | 4 | 2.25 | (0.720) | 5 | 2.72 | (1.497) | 4 | 1.92 | (0.777) | 5 | 2.74 | (0.751) |
| AR Cₘₐₓ | 4 | 2.44 | (1.028) | 5 | 2.17 | (1.138) | 5 | 1.87 | (0.801) | 5 | 2.41 | (0.996) |

AR = Accumulation Ratio (Day 21/Day 1)
Day 15 trough levels not collected for Cohort 1
Day 1 t_{1/2} not calculable

The PK after a single dose of BTX 1503 5% (w/w) showed that increased dosing (volume and/or frequency) resulted in increased plasma levels of BTX 1503. CBD levels were first observed between 2 and 3 hours after initial dosing. After the first dose (QD or BID), the AUC₍₀₋₂₄₎ increased from 7.13 h*ng/mL in Cohort 1 to 11.69 h*ng/mL in Cohort 4. The mean maximum plasma concentration (Cmax) after the first dose (QD or BID) increased from 0.35 ng/mL in Cohort 1 to 0.89 ng/mL for Cohort 4. Tₘₐₓ occurred at 18 hours (Cohort 1) and 10 hours (Cohort 2) after QD dosing and at 19 to 20 hours (after the first dose was administered) for BID dosing. Levels of CBD were below the limits of quantitation (BLOQ; <0.2 ng/mL) for all participants in Cohorts 3 and 4 by study Day 8, seven days after the initial single dose. Day 8 levels were not captured for Cohorts 1 and 2. The AUC and Cₘₐₓ increases from Cohort 1 to Cohort 4 did not increase proportional to dose suggesting a depot effect in the skin. The half-life (t_{1/2}) could not be calculated after the single dose.

During the multiple-dose phase of the study, mean trough levels on Day 15 did not show a clear dose effect. Day 15 plasma levels were not obtained for Cohort 1. The mean CBD trough level for Cohort 2 was 0.781 ng/mL, Cohort 3 was 0.525 ng/mL, and Cohort 4 was 2.11 ng/mL. There was one outlier in Cohort 4 that significantly skewed the mean levels (5.99 ng/mL). Without this participant, the mean trough level on Day 15 for Cohort 4 was 1.16 ng/mL.

By Day 21, CBD levels appeared to be at steady state as the second daily dose in the BID cohorts, Cohort 2 and Cohort 4, did not meaningfully elevate the CBD levels. In addition, the mean pre-dose levels on Day 21 (0.545, 0.770, 0.715, and 1.553 ng/mL for each cohort, respectively), were not elevated above the Day 15 trough levels (i.e., trough levels did not differ between the 7^{th} and 14^{th} dose). The maximum mean AUC₍₀₋₄₈₎ was 63.87 h*ng/mL and the maximum mean Cₘₐₓ was 2.17 ng/mL both occurring in the highest dose cohort, Cohort 4. The Accumulation Ratios (AR; Day 21/Day1) for AUC₍₀₋₂₄₎ were consistent for all cohorts ranging from 1.92 to 2.74 indicating that there was limited accumulation. CBD plasma levels dropped dramatically between 24 to 48 hours after the final dose, but did not return to zero. The t_{1/2} after Day 21 dosing was highly variable ranging from 22.76 (±8.669) hours in Cohort 2 to 66.79 (±67.914) in Cohort 1.

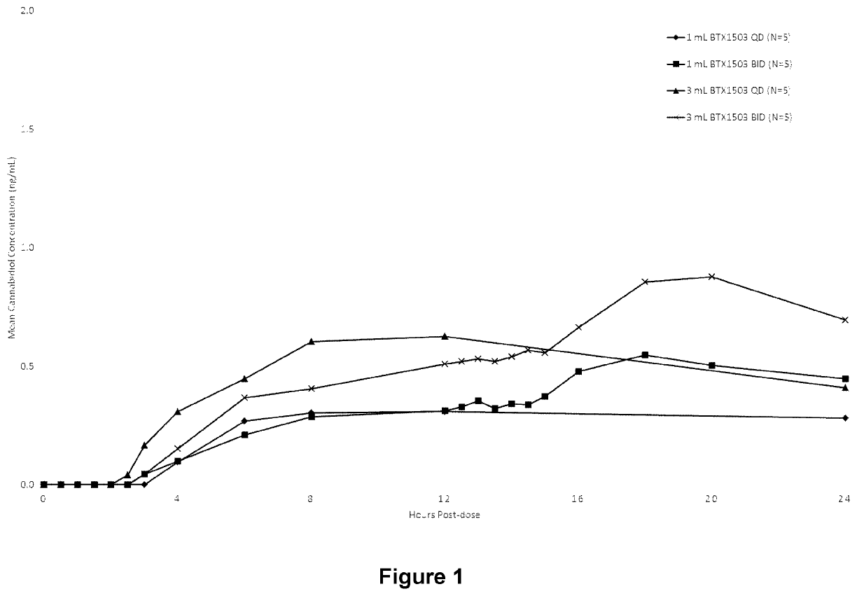

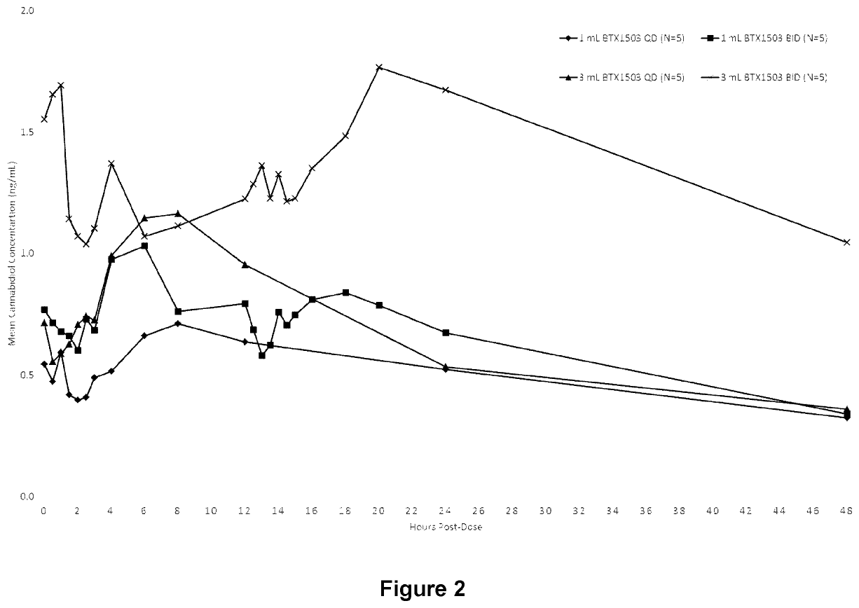

The mean CBD plasma concentrations displayed on the linear scale for each cohort are shown for Day 1 in FIG. 1 and for Day 21 in FIG. 2.

The PK of CBD after a single-dose and after 14 days of either QD or BID dosing with BTX 1503 5% Solution demonstrated that systemic absorption was low and dose dependent, although not dose proportional. CBD levels were first observed between 2 and 3 hours after initial dosing. Tmax occurred at 18 hours (Cohort 1) and 10 hours Cohort 3) after QD dosing and at 19 to 20 hours (after the first dose was administered) for BID dosing. Levels of CBD were below the limits of quantitation (BLOQ; <0.2 ng/mL) for all participants in Cohorts 3 and 4 by study Day 8, seven days after the initial single dose. By Day 21, CBD levels appeared to be at steady state. The mean maximum AUC(0-48) and Cmax at the end of the multiple-dose phase were 63.87 (±30.483) h*ng/mL and 2.17 (±1.209) ng/mL, respectively. The Accumulation Ratios (for AUC(0-24) were consistent for all cohorts ranging from 1.92 to 2.74 indicating that there was limited accumulation.

### Summary:

A total of 20 participants received study drug (5 per cohort) at a single site in Australia. Five participants in each of four cohorts completed their single-dose and multiple-dose (14 days) phase of the study.

This study demonstrated that daily topical treatment with up to 3 mL BID of BTX 1503 5% Solution (225 mg CBD per day) was safe and well tolerated. No participants discontinued the study. There were no SAEs reported. No AEs resulted in discontinuation or modification of study drug dosing. Forty-two AEs were reported in 18 of the 20 participants and all AEs, but one (moderate unrelated vasovagal reaction) were reported as mild. There did not appear to be a dose relationship with the reported AEs.

Facial dryness was most frequently reported treatment-related AE. Other AEs reported as at least possibly related included facial itchiness, erythema, nausea, stinging, and stinging in the eyes.

Cutaneous tolerability assessments showed one report of slight (“1”) erythema at the final evaluation, one report of slight (“1”) burning/stinging on Day 1, and seven reports of slight (“1”) dryness observed in two participants. There did not appear to be a dose relationship with cutaneous tolerability.

There were no clinically relevant changes from baseline observed in safety laboratory assessments (CBC, chemistry, and urinalysis), or in vital signs (blood pressure, temperature and pulse). No THC was observed in urine drug tests.

The pharmacokinetics of CBD after a single-dose and after 14 days of either QD or BID dosing with BTX 1503 5% Solution demonstrated that systemic absorption was low and dose dependent, although not dose proportional. Steady state levels were observed with 14 days of dosing and there was limited accumulation.

In this study of 20 healthy volunteers, treatment with 4 escalating doses of topically applied BTX 1503 5% (w/w) was shown to be safe and well tolerated at doses up to 225 mg daily. No serious or severe AEs were reported in any cohort and no participants withdrew from the study due to an AE. Facial dryness was the most frequently reported treatment-related TEAE. Daily cutaneous tolerability assessments showed that BTX 1503 was well tolerated with facial dryness rarely reported and only one report of mild erythema.

PK demonstrated that CBD was observed systemically at low concentrations after topical application. Systemic levels of CBD are at steady state levels after 28 days of dosing and were observed up to 48 hours after the final application.

### Example 7

An Open-Label Phase 1 b Study to Evaluate the Safety and Tolerability of BTX 1503 Solution in Patients with Acne Vulgaris. The object of this study is to determine the safety, tolerability, and pharmacology of BTX 1503 5% Solution in participants with acne vulgaris of the face.

Safety will be the primary outcome measure. The safety outcome measures to be assessed are:
Adverse events (AEs) will be monitored from time of consent through the end of study.
Cutaneous tolerability (erythema, scaling, dryness, burning/stinging, and irritant/allergic contact dermatitis) will be collected at Baseline, Day 14, Day 28, and Day 35 and graded using the following scale: 0, None; 1, Slight; 2, Moderate; 3, Severe.
Vital signs (temperature, blood pressure, and pulse) will be obtained at Baseline, Day 14, Day 28 and Day 35.
Complete blood count (CBC), chemistry, and urinalysis will be conducted at Baseline and at Day 28.
Urine drug tests for Tetrahydrocannabinol (THC) levels will be conducted at the Day 1, Day 28 and Day 35 Visits to evaluate for levels of THC

Blood levels of study drug will be measured.

Assessments of pharmacology will be evaluated by the treating dermatologist(s) through collection of lesion counts and Investigator Global Assessment (IGA) scores at Baseline, Day 28, and Day 35. Photographs will be obtained at Baseline, Day 28, and Day 35. An independent group of dermatologists will also review the photographs for IGA scoring. On Day 28 a Patient Reported Outcome (PRO) instrument will assess the participant's perception of the change in their acne relative to baseline.

### Methods

Participants will begin screening to determine eligibility to participate in the study. At the Screening Visit, informed consent, medical history, demographics, vital signs, height and weight will be obtained. A urine drug screen (UDS) will be performed. In addition, lesion counts on the face and an IGA will be conducted to assess participant eligibility.

If a participant is deemed eligible, they may be enrolled and begin Baseline assessments (within 14 days after the Screening Visit). Assessments for safety (CBC, chemistry, urinalysis, and vital signs) will be obtained at the Baseline Visit (Day 1). If the Screening and Baseline Visits are not conducted on the same day, lesion counts on the face, an Investigator's Global Assessment (IGA) for facial acne and a UDS will be repeated. Baseline photographs of the face and a blood sample for Baseline study drug plasma levels will be obtained. Clinical site staff will apply the first dose of study drug and participants will be observed in the clinic for one hour after application on Day 1. Cutaneous tolerability assessments will be conducted at one hour after the first application. Participants will be given two weeks of study drug and instructed in the proper application to cover their entire face twice daily.

On Day 7, a call will be made to each participant to ensure that they continue with dosing per instructions.

Participants will return to the clinic on Day 14 for vital signs, cutaneous tolerability assessments, and a blood draw for study drug plasma levels.

Participants will also be queried for AEs and changes in concomitant medications. Diaries and study drug will be returned and reviewed for compliance. In addition, the participant will apply their morning dose of study drug during the visit for the clinical site to confirm correct application techniques. Another 14 days of study drug will be dispensed along with the diary for the last two weeks of study drug treatment.

Participants will return to the clinic on Day 28 for safety assessments; vital signs, AEs, blood samples for CBC, chemistry and drug levels, and urine sample for urinalysis. Cutaneous tolerability assessments will also be obtained at the Day 28

Lesions counts will be collected by the clinical site along with photographs of the participant's face. Inflammatory and non-inflammatory lesion counts will be made separately. The IGA will be conducted by the study investigator at each site. Each participant will have the IGA done by the same investigator throughout the study. IGA will also be evaluated by the central panel through review of photographs.

Demographics will be summarised by age, gender, race, ethnicity height and weight. Summary statistics will be prepared for the change from baseline in lesion counts (inflammatory and non-inflammatory separate and combined) and IGA separately for the investigators and the central panel (IGA only). For continuous variables, the mean, standard deviation (SD), median, and range will be presented along with the 95% confidence interval (CI). Categorical variables will be summarised by proportions along with the 95% CI.

Summary statistics will be prepared for the change from baseline in lesion counts (inflammatory and non-inflammatory separate and combined) and IGA for both investigator(s) and the central panel (IGA only).

Lesion counts and changes in lesion counts will be presented. The following exploratory analyses will be conducted:
The absolute and percent change from baseline in the inflammatory lesion count at Day 28 and 35
The absolute and percent change from baseline in the non-inflammatory lesion count at Day 28 and 35
The absolute and percent change from baseline in the total lesion count at Day 28 and 35
The proportion of participants with an IGA score of “clear” or “almost clear” and at least a 2-grade reduction at Day 28 and 35

The participant's assessment of the change in their acne from baseline to Day 28 using the PRO assessment will be summarized by the proportion of subjects reporting each category (Much better, Slightly better, The same, Slightly Worse, Much worse).

Participants will return to the clinic one week following their final dose for a final blood draw for CBC, chemistry and study drug levels, urinalysis, cutaneous tolerability assessments, and AEs. Urine drug tests will be conducted at the Day 28 Visit. Lesion counts on the face and an IGA for facial acne will be conducted. Photographs of the face will be obtained.

Adverse events and concomitant medications will be monitored throughout the study. See Table 2 for full Schedule of Assessments.

In the proposed Phase 1b study, patients with moderate to severe acne would receive the same dose identified above for 28 consecutive days. This dose level is well below that tested and shown to be well-tolerated in a 28-day study previously carried out by the present laboratory in minipigs. Specifically, the NOAEL for dermal tolerability of BTX 1503 5% (w/w) on the skin of minipigs was 3.0 mg/cm2/day (150 mg/kg/day), which is ˜7.5 times the daily dose proposed in the Phase 1b study. In addition, based on the ratio of the mean Cmax observed in the 28-day minipig study to the mean Cmax in the 3 mL BID cohort in the Phase 1a study, there was >300 times the level of CBD, with no observed effect, in the minipigs.

Therefore, the dose level proposed for the Phase 1 b study is lower or identical to that has previously been shown to be well-tolerated in both nonclinical and clinical studies for BTX 1503 5% Solution.

In the present study, each milliliter of the BTX 1503 5% Solution contains 37.5 mg of CBD. Participants will apply 3 mL of the BTX 1503 5% Solution twice daily resulting in a maximum of 225 mg of CBD applied to the face daily. Participants will receive BID application of study drug for 27 days with a final application on the morning of Day 28 for a total of 55 doses.

### Statistical Methods

All statistical processing will be performed using SAS® unless otherwise stated.

Safety Analyses: All participants who receive at least one confirmed dose of study drug, and have at least one post-Baseline assessment will be included in the safety analyses.

Concomitant medication will be mapped to ATC Level 2 using the WHODrug dictionary. The number and percentage of participants reporting each medication will be summarised. Medications taken by each participant will be listed.

Cutaneous tolerability scores for each parameter (erythema, scaling, dryness, burning/stinging, and irritant/allergic contact dermatitis) will be summarised for each visit. In addition, the change from baseline in the mean scores will be summarised for each visit.

### Drug Levels:

Blood levels of study drug will be summarised by time point. The mean, SD, median and range will be presented.

### Sample Size:

The selected sample size is based on having appropriate sensitivity to observe a safety signal in participants with acne vulgaris. Sixteen participants receiving active BTX 1503 5% Solution will be adequate to detect if there are any cutaneous or systemic safety or tolerability concerns.

This will be a multi-center, randomized, double-blind, vehicle-controlled, parallel group, dose-finding study.

### Example 8

A Randomized, Double-Blind, Vehicle-Controlled Phase 2 Study to Evaluate the Safety and Efficacy of BTX 1503 in Patients with Acne Vulgaris. The objective of this study will be to determine the safety and efficacy with 84 days of treatment with BTX 1503 5.0% BID or QD or BTX 1503 2.5% QD compared to Vehicle BID or QD in subjects with moderate to severe acne vulgaris of the face.

This will be a multi-center, randomized, double-blind, vehicle-controlled, parallel group, dose-finding study.

### Outcome Measurements:

The efficacy outcome measures are:
Counts of inflammatory and non-inflammatory lesions at Baseline, Day 28, Day 56, Day 84, and at the two-week follow-up,
Investigator Global Assessment (IGA) scores at Baseline, Day 28, Day 56, Day 84, and at the two-week follow-up,
The Acne-QoL at Baseline and Day 84, and
A Patient Reported Outcome (PRO) instrument at Day 84 assessing the subject's perception of the change in their acne relative to Baseline.

The safety outcome measures to be assessed are:
Adverse events (AEs) monitored from time of consent through the end of study.
Cutaneous tolerability (erythema, scaling, dryness, burning/stinging, and irritant/allergic contact dermatitis) collected at Baseline, Day 28, Day 56, Day 84 and at the two-week Follow-up Visit and graded using the following scale: 0, None; 1, Slight; 2, Moderate; 3, Severe.
Complete blood count (CBC), chemistry, and urinalysis conducted at Baseline and at Day 84.

### Sample Size

Subjects will be randomized 2:2:2:1:1 (BTX 1503 5% BID:BTX 1503 5% QD:BTX 1503 2.5% QD:Vehicle BID:Vehicle QD) with 90 subjects in each BTX 1503 group and 45 subjects in each vehicle group for a total of 360 subjects.

### Eligibility Criteria:

To be included in the study, subjects with acne vulgaris must meet the following inclusion criteria:
either gender between 12 and 45 years of age, inclusive.
good general health without clinically significant haematological, cardiac, respiratory, renal, endocrine, gastrointestinal, psychiatric, hepatic, or malignant disease, as determined by the investigator.
acne vulgaris of the face defined as:
   a. 20 to 50 (inclusive) inflammatory lesions on the face
   b. 20 to 100 (inclusive) non-inflammatory lesions on the face
   c. An Investigator Global Assessment (IGA) score for acne severity of 3 or 4 (moderate or severe) assessed on the face.
≤3 nodular/cystic acne lesions (>5 mm in diameter)

### Methods

If a subject is deemed eligible, they may be enrolled and begin Baseline assessments (within 14 days after the Screening Visit). Assessments for safety (CBC, chemistry, and urinalysis) will be obtained at the Baseline Visit (Day 1). If the Screening and Baseline Visits are not conducted on the same day, lesion counts on the face, an IGA for facial acne and a UDS will be repeated. Baseline photographs of the face (selected sites) will be obtained. The Acne-QoL will be administered. Clinical site staff will apply the first dose of study drug. Cutaneous tolerability assessments will be conducted 10 minutes after the first application. Subjects will be given 28 days of study drug and instructed in the proper application to cover their entire face.

Subjects will return to the clinic on Day 28 for cutaneous tolerability assessments, lesion counts and IGA. Subjects will also be queried for AEs and changes in concomitant medications. Diaries and study drug will be returned and reviewed for compliance. In addition, the subject will apply their morning dose of study drug during the visit for the clinical site to confirm correct application techniques. Another 28 days of study drug will be dispensed along with the diary for the next 28 days of study drug treatment.

Subjects will return to the clinic on Day 56 for cutaneous tolerability assessments, lesion counts and IGA. Subjects will also be queried for AEs and changes in concomitant medications. Diaries and study drug will be returned and reviewed for compliance. In addition, the subject will apply their morning dose of study drug during the visit for the clinical site to confirm correct application techniques. Another 28 days of study drug will be dispensed along with the diary for the next 28 days of study drug treatment.

Subjects will return to the clinic on Day 84 for safety labs (CBC, chemistry, and urinalysis). Cutaneous tolerability assessments and AEs will also be obtained at the Day 84 Visit as will recording of concomitant medications. Lesion counts on the face and an IGA for facial acne will be conducted. Photographs of the face will be obtained at selected sites. The Acne-QoL will be administered along with the PRO instrument assessing the subject's perception of the change in their acne relative to baseline.

Subjects will return to the clinic two weeks following their final dose for the Follow-up Visit. If any abnormal lab findings were observed at Day 84, a blood draw for CBC and chemistry and urine for urinalysis will be collected. Cutaneous tolerability assessments and AEs will also be obtained at the two-week Follow-up Visit as will recording of concomitant medications. counts on the face and an IGA for facial acne will be conducted.

Adverse events and concomitant medications will be monitored throughout the study.

### Statistical Method

All statistical processing will be performed using SAS® 9.3 or higher. Demographics will be summarized by age, gender, race, ethnicity, height and weight. Summary statistics will be presented for change from baseline in lesion counts (inflammatory and non-inflammatory separate and combined) and IGA. For continuous variables, the mean, standard deviation (SD), median, and range will be presented along with the 95% confidence interval (CI). Categorical variables will be summarized by proportions along with the 95% CI.

### Analysis Sets:

This study will be evaluated using 3 analysis sets: intent-to-treat (ITT), per protocol (PP), and safety. Efficacy conclusions will be drawn from the ITT analysis set. The PP analysis set will be used to support the efficacy findings in the ITT analyses. Safety conclusions will be drawn from the safety analysis set.

The ITT analysis set includes all subjects who are randomized and provided with study drug and is based on randomized study group, regardless of study drug received. The safety analysis set includes all subjects who are randomized, receive at least 1 confirmed dose of study drug, and have at least 1 post-Baseline assessment. The safety analysis set will be assessed based on study drug received, regardless of group to which subject was randomized. The PP analysis set includes all subjects in the ITT analysis set who complete the Day 84 evaluation without noteworthy study protocol violations.

Subjects who have a documented lack of treatment effect or who are discontinued from the study due to an AE that was considered by the investigator to be study drug related will be included in the PP analysis set. Specific criteria for identifying the PP analysis set will be determined prior to breaking the blind.

Vehicle QD and Vehicle BID groups will be combined for analyses.

### Efficacy Analyses:

The efficacy analyses will be performed using the ITT (primary) and PP (supportive) analysis sets. The efficacy variables include the IGA and lesion counts (inflammatory and non-inflammatory) collected at Screening/Baseline and all subsequent study visits. Absolute and percent changes in lesion counts from Baseline will be calculated for each subject at study Days 28, 56, and 84 and at the two-week follow-up. The IGA will be dichotomized into “success” and “failure” at study Days 28, 56, and 84 and at the 2-week follow-up, with a subject considered a “success” at each individual visit if the IGA at that visit is Clear (“0”) or Almost Clear (“1”) and at least 2 grades less than the Baseline score. Efficacy variables also include the Acne-QoL which will be scored accordingly to the authors scoring system (Martin 2001), and the subject's assessment of improvement (PRO) using proportions by category.

Descriptive statistics (including mean, median, standard deviation [SD], minimum, and maximum, unless otherwise stated) will be presented for the following parameters by study group using both the ITT and PP analysis sets:
Inflammatory and non-inflammatory lesion counts at Baseline and study Days 28, 56, 84 and the two-week follow-up.
Absolute and percent change from Baseline in inflammatory and non-inflammatory lesion counts at study Days 28, 56, 84 and the two-week follow-up.
Frequency and percent distribution of the dichotomized IGA at study Days 28, 56, 84 and the two-week follow-up.

### Primary Endpoint:

The primary efficacy endpoint for the study is:
Absolute change from Baseline to Day 84 in inflammatory lesion counts.

### Secondary Endpoints:

The secondary endpoints for the study are:
Absolute change from Baseline to Day 84 in non-inflammatory lesion count, and
The proportion of subjects with an IGA score of “clear” or “almost clear” at Day 84 and at least a 2-grade reduction from the Baseline IGA score
The percent change from Baseline in the inflammatory lesion count at Day 84,
The percent change from Baseline in the non-inflammatory lesion count at Day 84, Day 84 (PRO).
Absolute and percent change from Baseline in inflammatory and non-inflammatory lesion counts at the Follow-up visit.
The proportion of subjects with an IGA score of “clear” or “almost clear” and at least a 2-grade reduction from the Baseline IGA score at the Follow-up visit

This Phase 2 study is designed to identify the response to two different concentrations and dosing frequency of BTX 1503. Statistical tests applied to the outcomes will be exploratory for establishing the dose and sample size for Phase 3 studies. No adjustments for Type 1 error will occur.

Tests of superiority for the primary and secondary endpoints of absolute change from Baseline in lesion counts will be based on either parametric or nonparametric methods consistent with the statistical assumptions required to support the analyses. Specifically, the tests of superiority will be based on an ANCOVA with factors of treatment and the respective Baseline lesion count as a covariate, or on ranked data submitted to an ANCOVA with factors of treatment and analysis center and the respective Baseline lesion count as a covariate. If the treatment-by analysis center interaction effect is significant at an alpha less than 0.10, then the effect will be included in the model; otherwise it will be removed.

A skewness test will be applied to the residuals resulting from an ANCOVA. A 2-sided p-value for the skewness test significant at 0.01 will lead to the use of the nonparametric method. If a parametric analysis is indicated, the results of the parametric analysis will be considered the primary analysis. If a nonparametric analysis is indicated, the absolute or percent changes in inflammatory lesion counts will be rank-transformed prior to submitting them to the ANCOVA. Results of the rank-transformed analyses will then be considered the primary analysis; results of the nonrank-transformed analyses will be also presented.

The analysis of the dichotomized IGA will be based on a Cochran-Mantel-Haenszel (CMH) test at Day 84. Pairwise tests will be conducted comparing the treated groups to vehicle. The lesion count and IGA analyses employ the previously described methods for exploratory endpoints. The exploratory endpoints are:
change from Baseline in the total lesion count at Day 84,
percent change from Baseline in the total lesion count at Day 84,
change from Baseline in the Acne-QoL at Day 84, and
subject's assessment of the change in their acne from baseline.

### Subset Analyses

Subset analyses will be conducted for the primary efficacy endpoint. These analyses will be summarized using descriptive statistics. The specific subsets within the ITT analysis set that will be evaluated include: Baseline IGA, sex, age, ethnicity, and race. Additionally, the efficacy variables will be evaluated for the group less than the median age and greater than or equal to the median age.

### Safety Analyses:

All subjects who receive at least one confirmed dose of study drug, and have at least one post-Baseline assessment will be included in the safety analyses.

Concomitant medication will be mapped to ATC Level 2 using the WHODrug dictionary. The number and percentage of subjects reporting each medication will be summarized. Medications taken by each subject will be listed.

Cutaneous tolerability scores for each parameter (erythema, scaling, dryness, burning/stinging, and irritant/allergic contact dermatitis) will be summarized for each visit. In addition, the change from baseline in the mean scores will be summarized for each visit.

### Example 9

Study of skin permeation and delivery measurements from cannabidiol formulations. The primary objective of the study was to determine the rate and extent of in vitro skin permeation of cannabidiol (the “Active”) into and through cadaver skin using a Franz diffusion cell system. Flux was measured over a period of 48 hours after application of the formulations.

TABLE 6
Formulations
Formulation

A: 2.5 wt % cannabidiol
B: 5.0 wt % cannabidiol
C: 2.5 wt % cannabidiol
D: 5.0 wt % cannabidiol

Intact human cadaver skin was purchased from the New York Firefighter's Skin Bank (“NYFFSB”, NY, N.Y.). The skin tissue was dermatomed by the tissue bank to a thickness of some 250 μm and shipped frozen on dry ice. Upon receipt of the donor skin, the skin pieces were stored at −20° C. until used. Prior to use, the skin pieces were removed from the freezer and allowed to thaw fully at ambient temperature.

The following equipment was used during the course of the study:
Diffusion Cells. 24 diffusion cells with 3.3 ml receptor volume and a 0.55 cm² receptor fluid exposure surface area.
Stirring Dry Block Heaters. Reacti-Therm #18823 stirring dry block heaters were used to maintain the receptor fluid at 32±0.5° C. with constant stirring throughout the study.
The analysis was carried out with an Agilent 1260 HPLC unit with a G16120 MS detector, ID #: TM-EQ-069.
Tritiated water signals were analyzed with a PerkinElmer MicroBeta TriLux 1450 Liquid scintillation counter (“LSC”). ID #: TM-EQ-047.

The following materials and reagents were used for the study.

TABLE 7
Materials and reagents used in the study

| Material | Supplier: | Catalog#: | Lot#: |
|---|---|---|---|
| Cannabidiol | Botanix | | 9100042 |
| Methanol | FisherSci Optima | A456-4 | 173438 |
| Water | Millipore | WX0008-1 | 56160 |
| Hydroxypropyl-β- | TCI | H0979 | PJT4B |
| cyclodextrin (HPBCD) | | | |
| Brij O20 | Croda | 436240 | MKBP0994V |
| Formic Acid | Sigma Aldrich | 56302 | BCBQ3264V |
| Ammonium formate | Sigma Aldrich | 17843 | BCBP1919V |
| ³H Water | Perkin Elmer | Net001B001 | 1738956 |
| PBS (10X diluted to | Quality | 119-069-131 | 721676 |
| 1X) | Biologicals | | |
| Zorbax Eclipse PAH | Agilent | | |
| narrow bore C18 RR | | | |
| (2.1 × 100 mm, | | | |
| 3.5 um, 600 bar) | | | |

A liquid chromatography mass spectrometry (“LC/MS”) analytical method was used to detect for cannabidiol (“CBD”).

### Preparation of Mobile Phases

Mobile Phase A: Mobile Phase A was prepared by first transferring 1.0 ml of formic acid (Sigma Aldrich: 56302) into a 2 L media bottle. 1 L of HPLC grade water (Millipore: WX0008-1) was then measured in a volumetric cylinder and the contents transferred into the 2 L media bottle. Finally, 630.6 mg of ammonium formate was then weighed and also transferred to the media bottle. The mixture in the media bottle was then shaken until the contents were fully dissolved. Mobile Phase A was stored for less than one week during the course of the analysis.

Mobile Phase B: Mobile Phase B was prepared by transferring 1.0 ml of Formic acid (Sigma Aldrich: 56302) into a 2 L media bottle. 1 L of HPLC grade methanol (Millipore: AX-0145P) was then measured in a volumetric cylinder and the contents transferred into the 2 L media bottle. Finally, 630.6 mg of ammonium formate was then weighed and also transferred to the media bottle. The mixture in the media bottle was shaken until the contents were fully mixed. Mobile Phase B was stored for less than one week during the course of the analysis.

### Preparation of Stock Solution and Calibration Standards

Individual calibration standards were prepared for CBD. A CBD “Stock Solution” was prepared by first weighing 4 mg of CBD with an analytical balance in a glass vial. The vial was then tared on the balance and 4 ml of dimethyl sulfoxide (“DMSO”) was introduced in to the glass vial with a pipettor. The vial was reweighed. The vial was then removed from the analytical balance and capped. The capped vial was vortexed and sonicated using an ultrasonication bath until the CBD was fully dissolved.

The above procedure was used to make a 1 mg/ml Stock Solution for CBD. Further calibration standards were prepared through serial dilution. In each serial dilution, 3001 of the preceding calibration standard was diluted with 1200 μl of DMSO. Eight calibration standards were prepared. The CBD concentration in each of the calibration standards is shown in Table 8 below.

TABLE 8
Calibration standards and the corresponding
concentration of the CBD.

| Active | CBD | |
|---|---|---|
| Calibration standard | Conc (μg/ml) | |
| Stock Solution | 1000 μg/ml Stock Solution | |
| Cal 2 | 200 | μg/ml |
| Cal 3 | 40 | μg/ml |
| Cal 4 | 8 | μg/ml |
| Cal 5 | 1.6 | μg/ml |
| Cal 6 | 0.32 | μg/ml |
| Cal 7 | 0.064 | μg/ml |
| Cal 8 | 0.0128 | μg/ml |

The CBD was first prepared in a Stock Solution. Separate calibration standards were then prepared for by serial five-fold dilutions with DMSO. Standards Cal3-Cal8 were used for the calibration curves.

### Preparation of Sample Solution

The study samples were collected during the permeation studies. No further preparation was done on the samples prior to analysis.

### Chromatographic Parameters

An outline of the method details is provided in Table 9 below.

TABLE 9
Chromatographic parameters for CBD detection.

| Instrument: | 1200-HPLC/UV/MSMS Xevo TQD | | |
|---|---|---|---|
| Column: | Zorbax Eclipse PAH narrow bore C18 RR (2.1 × | | |
| | 100 mm, 3.5 um, 600 bar) | | |
| | Guard column: PAH 12.5 × 3.5 μm | | |
| Mobile phase: | A: Water with 0.1% FA, 10 mM NH₄HCO₂ | | |
| | B: Methanol with 0.1% FA, 10 mM NH₄HCO₂ | | |
| Gradient: | Time (minutes) | % B | |
| | 0 | 70% | |
| | 1.0 | 70% | |
| | 5.0 | 95% | |
| | 7.0 | 95% | |
| | Post time | 3 | min |
| Flow rate: | 1.0 ml/min | | |
| Column | 50° C. | | |
| temperature: | | | |
| MS detection: | ESI(+)-MRM: m/z 315.2 > 193.1 | | |
| | Collision energy: 20 V | | |
| Injection volume: | 5 μl | | |
| Diluent for | DMSO | | |
| standards: | | | |
| Retention time: | CBD | ~4.2 | minutes |

### Calculation

After the LC/MS testing was complete, the samples were analyzed using Chemstation software. The AUCs of the CBD peaks were recorded and converted to g/ml values using a calibration curve developed from the calibration standards' AUC values and known concentration values. These μg/ml values were imported into the study results Excel workbook. These concentrations were then multiplied by the receptor volume (3.3 mL) and divided by the surface area of the skin exposed to the receptor fluid (0.55 cm²) for an end cumulative amount in μg/cm². For receptor fluid time points greater than 4 hrs, this μg/cm² value was corrected for the sample aliquot volumes which were removed to compensate for the dilution caused by replacing the sample volume with fresh buffer solution. As an example, for the second time point at 10 hrs, the dilution factor (300 μl aliquot/3.3 ml receptor volume or 1/11) is multiplied by the μg/cm² value calculated for the 4 hr time point, the result of which is then added to the μg/cm² concentration which is calculated using the 10 hr AUC value. Equation 1 outlines the correction value for the dilution effect.

$$Equation \#1 A ( Dilution correction )$$

$$Cumulative amount ( \text{?} ) = \frac{\begin{matrix}( \text{?} + \sum ( \text{?} of previous \text{?} ) \times \frac{sample volume}{receptor volume} ) \times \\ receptor volume\end{matrix}}{( calibration \text{?} \times surface area )}$$

$$\text{?} \text{indicates text missing or illegible when filed}$$

### Receptor Fluid

The receptor fluid (the “Receptor Fluid”) consisted of phosphate buffered saline (“PBS”), sourced from Quality Biologicals with 0.01 wt % NaN₃ (added as a preservative), 4 wt % hydroxypropyl-□-cyclodextrin (added to increase solubility of the Actives) and 1 wt % Brij O20. The PBS was supplied as 10× concentration and was diluted to 1× concentration prior to the study by volumetrically adding distilled water at a 9:1 water to concentrated PBS ratio. The solubility of CBD in the Receptor Fluid was previously measured to be ˜>50 μg/ml and was determined to be sufficient to maintain sink conditions throughout the study.

After mixing the Receptor Fluid, degassing of the Receptor Fluid was accomplished according to Tioga's Standard Operating Procedure (“SOP”) SOP Lab.007.1 ‘Degassing of receptor fluid for diffusion studies’. Receptor Fluid was filtered through a ZapCap CR 0.2 μm membrane under vacuum; the Receptor Fluid, so filtered, was stirred for an additional 20 minutes under vacuum.

### Skin Preparation

Human cadaver skin from NYFFSB was prepared as follows prior to assembling the diffusion cells.
The cadaver skin piece was removed from the freezer and allowed to defrost in a Bio-safety hood for 30 minutes. Prior to opening the package, a visual inspection was used to confirm that the skin piece had been thoroughly defrosted.
The cadaver skin piece was removed from the package and placed in a distilled water bath for 30 seconds to wash off any cryoprotectants from the skin. The skin was then removed from the water bath and placed in a Bio-safety hood. The exterior surface of the skin was patted dry with a KimWipe, sprayed with fresh PBS, and then patted dry again.

### Assembling the Franz-Type Diffusion Cells

Glass FDCs with a 3.3 ml receiver volume and 0.55 cm² diffusional area, custom fabricated to Tioga specifications, were used. Once the skin had been defrosted and washed, the FDCs were prepared as follows:
The receptor wells were filled with degassed Receptor Fluid using a pipette.
A 6 mm by 3 mm diameter Teflon coated magnetic stir bar was introduced into each receptor well.
The defrosted and washed cadaver skin pieces were examined and only areas of even thickness and with no visible surface damage were used.
The skin piece was cut into approximately 2 cm×2 cm squares using skin scissors. The square sizes were adjusted as necessary according to the shape and dimensions of the skin piece, but were selected to be approximately uniform in size among all FDCs.
A skin piece was centered on each inverted donor compartment, with the stratum corneum (“SC”) side contacting the donor compartment.
The donor and receptor well compartments were then aligned and clamped together with a pinch clamp, ensuring that the skin pieces were centered between both donor and receptor wells.
Additional Receptor Fluid was added as necessary. Air bubbles in the receptor well, if any, were removed by tilting the FDC assembly such that the air escapes along the sample port. Receptor wells were filled with approximately 3.3 ml of Receptor Fluid.
The assembled FDCs were placed into stirring dry block heaters which were preheated to 32° C. The Receptor Fluid was continuously agitated via the magnetic stir bar.
After 20 minutes, the surface of the skin in each FDC was examined. If the skin appeared wet or showed signs of sweating, the cell was discarded.
Approximately 24 FDCs were assembled from the skin piece.

### Membrane Integrity Check

Once the FDCs were assembled, the barrier integrity of the skin pieces was tested prior to application of the test articles by a measurement of the transdermal flux of tritiated water according to Tioga SOP Lab.011.1, as outlined following:
Into 10 ml of deionized (“DI”) water was introduced 25 μl of 1 mCi/ml water (the resulting sample was termed “Tritiated Water”).
An aliquot of 150 μl of Tritiated Water was introduced into each FDC donor well.
After 10 minutes, the Tritiated Water was removed from each FDC donor well using a pipette and the skin surface tapped dry using a KimWipe.
The receptor well of each FDC was agitated for an additional 1 hour after the Tritiated Water had been removed from each donor well.
After the 1 hour of agitation, a 300 μl aliquot was abstracted from each FDC receptor well and placed into a well in a microtiter plate.
600 μL of scintillation cocktail (Ultima Gold from Perkin Elmer) was then added to each sample aliquot in the microtiter plate.
The tritium (³H) content of each sample aliquot was measured using a liquid scintillation counter (“LSC”—PerkinElmer MicroBeta TriLux 1450).
After LSC analysis was complete, results were analyzed. Any FDCs showing anomalously high water flux were discarded.
The remaining FDCs were ranked according to the magnitudes of the measured tritiated water flux values. Test articles were then assigned to the batch of FDCs such that the replicates for each test article are each applied to a skin piece with nearly equivalent average tritiated water flux values. The ranking of skin pieces was carried out separately for each substrate.
The entire volume of Receptor Fluid was removed from each FDC and replaced with fresh Receptor Fluid.
The FDCs were finally placed into preheated dry block heaters.

### Test Article Application Procedure

After the membrane integrity tests were complete and the cells appropriately sorted, samples of the test articles were then applied to the stratum corneum of the skin. A one-time dosing regimen was used for this study. Donor cells were left uncapped during the experiment. The dose of the Active applied per cell and corresponding formulation is shown in Table 10 below.

TABLE 10
CBD dose per cell for the applied test articles.

| Study | | Dose of formulation | |
|---|---|---|---|
| Arm | Formulation | applied per cell | CBD dose |
| 1 | A: 2.5 wt % cannabidiol | 5 μl | 173.9 μg/cm² |
| 2 | B: 5.0 wt % cannabidiol | 5 μl | 340.9 μg/cm² |
| 3 | C: 2.5 wt % cannabidiol | 5 μl | 173.9 μg/cm² |
| 4 | D: 5.0 wt % cannabidiol | 5 μl | 340.9 μg/cm² |

The dose assumes a specific gravity of 0.75 for the formulations, and also assumes 100% of the applied 5 μl of the formulation remains on the skin after spreading the formulation across the skin surface using a glass rod.

“Blank” undosed FDC cells were also set up to test for background signal noise. The background noise measured from these “blank” cells had negligible AUC for CBD.

### Sampling of Receptor Fluid

Using a graduated Hamilton type injector syringe, a 300 μl aliquot was abstracted from the sampling port of each FDC at each of 4, 10, 24 and 48 hours. Fresh Receptor Fluid was added to each receptor well to replace the volume of fluid abstracted. Each abstracted aliquot was introduced into a well in a 96-well microtiter plate.

Samples were stored in a refrigerator at 4-8° C. prior to LC/MS analysis. Samples were analyzed within 5 days of collection.

### Skin Extraction

At 48 hours, a 200 ul aliquot of 50 vol %/50 vol % water/ethanol was dispensed in the donor compartment of each FDC. This “washing solution” was allowed to sit for 5 minutes, after which it was removed. The skin was then tapped dry and tape stripped three times with cellophane tape, each tapestripping consisting of applying a piece of cellophane tape to the skin with light pressure and peeling off the tape, thereby systematically removing the upper most layers of the stratum corneum. The tape strips were discarded.

After tape stripping was complete, the remaining skin was split into epidermal and dermal compartments by using a pair of spatulas. If necessary, the skin was placed on a hot plate set at 60° C. for one minute to help facilitate the separation of the skin. The epidermal and dermal compartments were then separately placed into glass vials, into which 3 ml of DMSO was added to extract the CBD from the tissue. The skin pieces were then incubated at 40° C. for 24 hours with gentle agitation. After the 24 hour incubation period, samples were collected from the extraction solvent and analyzed via LC/MS detection.

### Analyses of Samples

The samples abstracted from the receptor wells were then analyzed using the MS method outlined above. The concentrations of CBD were assayed and reported in each case.

### Results

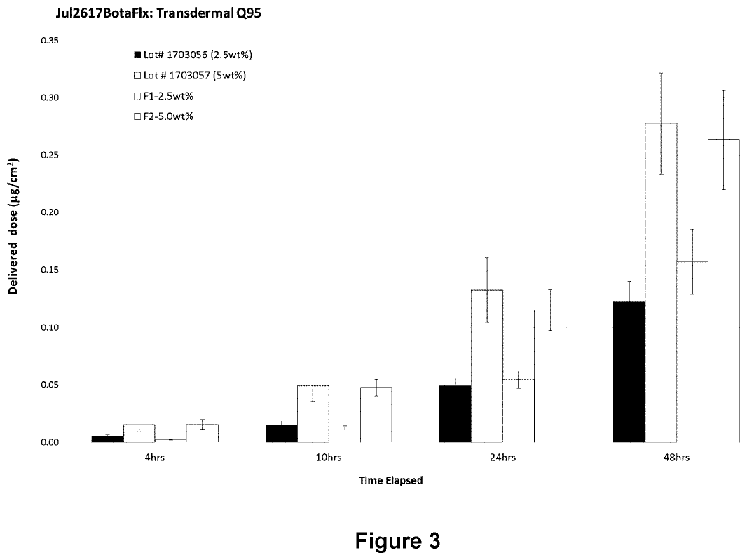

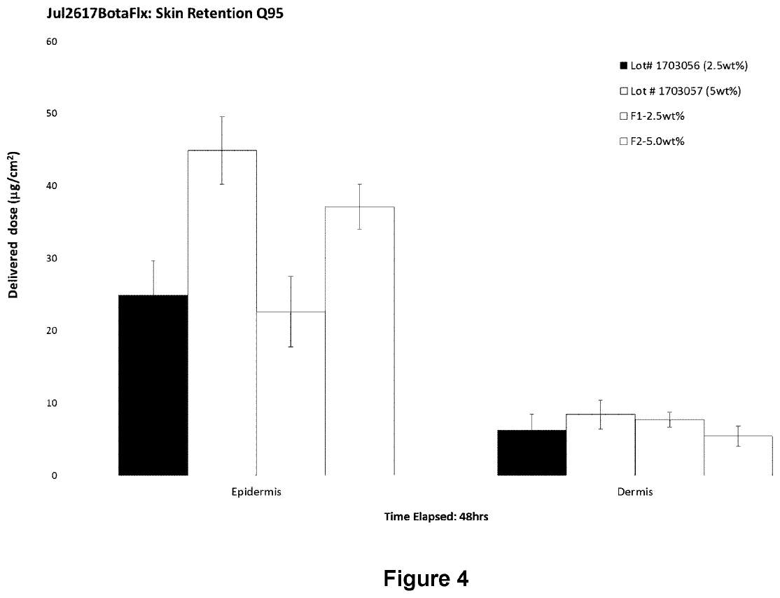

The accumulated dose of CBD at each of the time points is shown in FIGS. 3 and 4.

TABLE 11
Total accumulated dose (in μg/cm²) of CBD delivered over time.
Delivered dose (μg/cm²)

| | A: 2.5 wt % | B: 5.0 wt % | C: 2.5 wt % | D: 5.0 wt % |
|---|---|---|---|---|
| Time (h) | cannabidiol | cannabidiol | cannabidiol | cannabidiol |
| 4 | 0.005 | 0.015 | 0.022 | 0.016 |
| 10 | 0.015 | 0.049 | 0.013 | 0.048 |
| 24 | 0.049 | 0.133 | 0.055 | 0.115 |
| 48 | 0.122 | 0.278 | 0.157 | 0.263 |
| Epidermis | 24.921 | 44.884 | 22.641 | 37.156 |
| Dermis | 6.283 | 8.408 | 7.739 | 5.474 |
| Time (h) | Std Err | Std Err | Std Err | Std Err |
| 4 | 0.002 | 0.006 | 0.000 | 0.004 |
| 10 | 0.004 | 0.013 | 0.002 | 0.007 |
| 24 | 0.007 | 0.028 | 0.007 | 0.018 |
| 48 | 0.018 | 0.044 | 0.028 | 0.043 |
| Epidermis | 4.777 | 4.648 | 4.886 | 3.111 |
| Dermis | 2.187 | 2.023 | 1.055 | 1.395 |

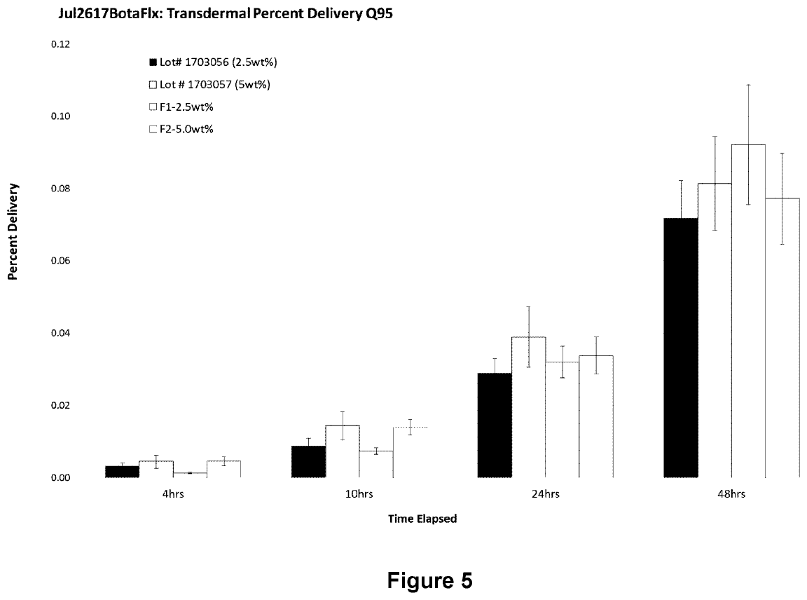

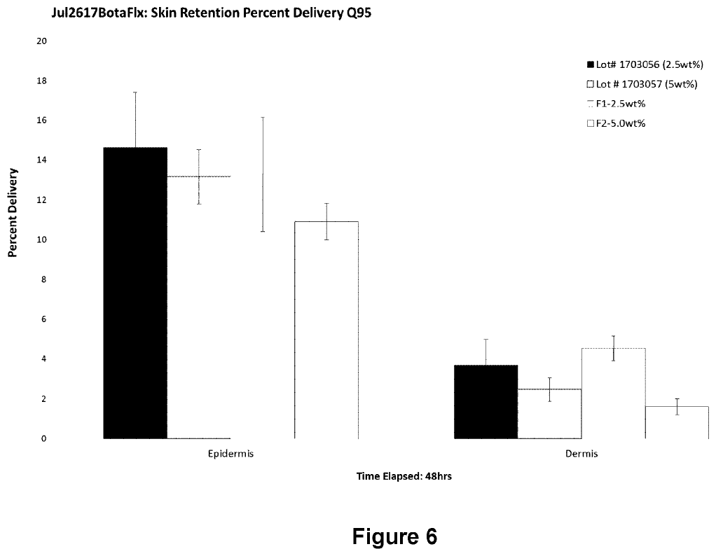

The percent delivery of CBD (taking into account the 5 μl applied dose and the formulated concentration of CBD in the formulation) at each of the time points is shown in FIGS. 5 and 6.

TABLE 12
Percent delivery of CBD delivered over time.
Percentage Delivery

| | A: 2.5 wt % | B: 5.0 wt % | C: 2.5 wt % | D: 5.0 wt % |
|---|---|---|---|---|
| Time (h) | cannabidiol | cannabidiol | cannabidiol | cannabidiol |
| 4 | 0.003 | 0.004 | 0.001 | 0.005 |
| 10 | 0.009 | 0.014 | 0.007 | 0.014 |
| 24 | 0.029 | 0.039 | 0.032 | 0.034 |
| 48 | 0.72 | 0.081 | 0.092 | 0.077 |
| Epidermis | 14.620 | 13.166 | 13.283 | 10.899 |
| Dermis | 3.686 | 4.540 | 7.739 | 1.606 |
| Time (h) | Std Err | Std Err | Std Err | Std Err |
| 4 | 0.001 | 0.002 | 0.000 | 0.001 |
| 10 | 0.002 | 0.004 | 0.001 | 0.002 |
| 24 | 0.004 | 0.008 | 0.004 | 0.005 |
| 48 | 0.010 | 0.013 | 0.017 | 0.013 |
| Epidermis | 2.802 | 1.363 | 2.866 | 0.913 |
| Dermis | 1.283 | 0.593 | 0.619 | 0.409 |

The percent delivery assumes a specific gravity of 0.75 and that 100% of the 5 μL applied dose remains on the skin after spreading the formulation with the glass rod. Percent delivery takes into account the varying concentrations of CBD present in each formulation.

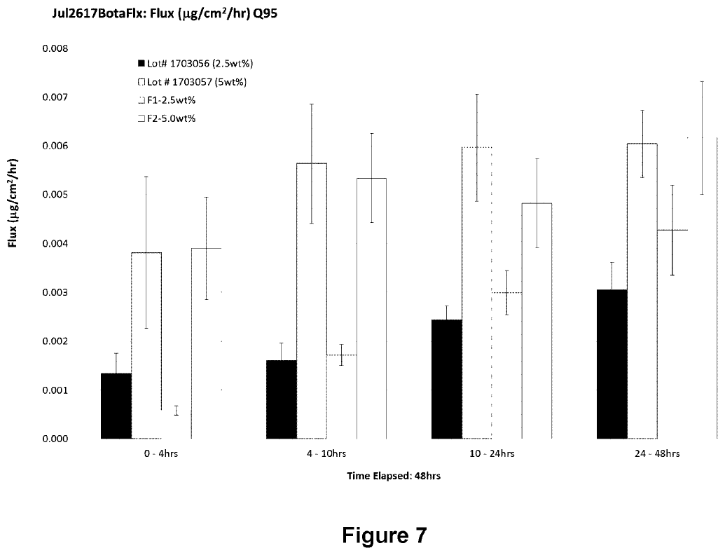

The flux of CBD between each of the time points is shown in FIG. 7.

TABLE 13
Flux of CBD overtime (in μg/cm2/hr).
Flux (μg/cm²/h)

| | A: 2.5 wt % | B: 5.0 wt % | C: 2.5 wt % | D: 5.0 wt % |
|---|---|---|---|---|
| Time (h) | cannabidiol | cannabidiol | cannabidiol | cannabidiol |
| 0-4 | 0.00134 | 0.00381 | 0.00058 | 0.00390 |
| 4-10 | 0.00161 | 0.00563 | 0.00172 | 0.00534 |
| 10-24 | 0.0244 | 0.00597 | 0.00300 | 0.00482 |
| 24-48 | 0.00305 | 0.00604 | 0.00427 | 0.00616 |
| Time (h) | Std Err | Std Err | Std Err | Std Err |
| 0-4 | 0.00041 | 0.00155 | 0.00010 | 0.00105 |
| 4-10 | 0.00035 | 0.00123 | 0.00022 | 0.00091 |
| 10-24 | 0.00028 | 0.00110 | 0.00045 | 0.00091 |
| 24-48 | 0.0056 | 0.00069 | 0.00092 | 0.00116 |

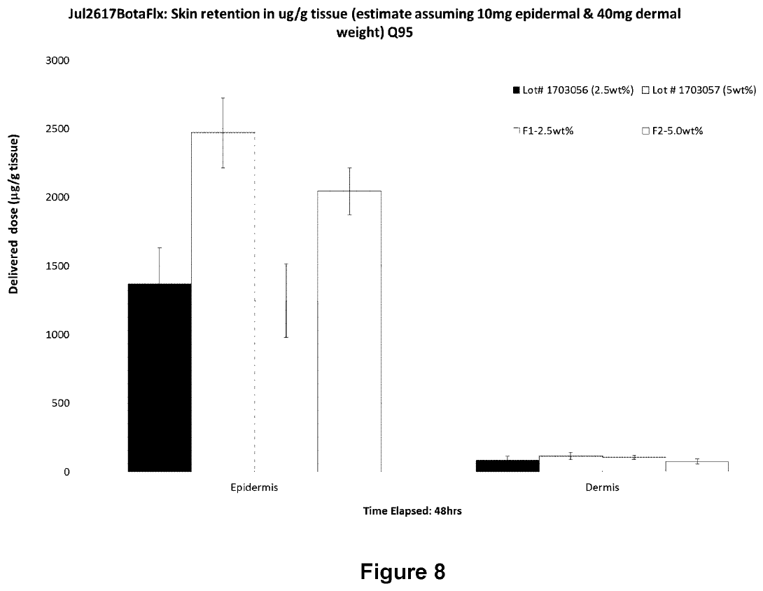

The accumulated dose of CBD in the epidermis and dermis was also calculated as μg of CBD delivered per gram of tissue. This calculation assumes a weight of 10 mg for the epidermal tissue and 40 mg for the dermal tissues (these values are based on average values observed in previous experiments). These values are shown in FIG. 8.

TABLE 14
Total accumulated dose in the skin (in μg/gram
tissue) of CBD delivered at 48 hrs.
Skin Delivery (μg/g)

| | A: 2.5 wt % | B: 5.0 wt % | C: 2.5 wt % | D: 5.0 wt % |
|---|---|---|---|---|
| Time (h) | cannabidiol | cannabidiol | cannabidiol | cannabidiol |
| Epidermis | 1370.66 | 2468.64 | 1245.24 | 2043.60 |
| Dermis | 86.39 | 115.60 | 106.41 | 75.26 |
| Time (h) | Std Err | Std Err | Std Err | Std Err |
| Epidermis | 262.71 | 255.62 | 268.71 | 171.11 |
| Dermis | 30.08 | 27.81 | 14.51 | 19.18 |

A two tailed Ttest with unequal variance was used to evaluate the CBD data sets over time. The Ttest compared the transdermal data sets at 24 hours and 48 hours and the epidermal and dermal values.

TABLE 15
A two-tailed Ttest with unequal variance was done comparing
the CBD data sets at 24 and 48 hrs, plus the epidermal
and dermal concentration (results shown are p-values).

| | | | | |
|---|---|---|---|---|
| | A: 2.5 wt % | B: 5.0 wt % | C: 2.5 wt % | D: 5.0 wt % |
| Formulation | cannabidiol | cannabidiol | cannabidiol | cannabidiol |

T Test for 24 h (probability values)

| | | | | |
|---|---|---|---|---|
| A: 2.5 wt % | 1 | | | |
| cannabidiol | | | | |
| B: 5.0 wt % | 0.040 | 1 | | |
| cannabidiol | | | | |
| C: 2.5 wt % | 0.613 | 0.049 | 1 | |
| cannabidiol | | | | |
| D: 5.0 wt % | 0.016 | 0.617 | 0.022 | 1 |
| cannabidiol | | | | |

T Test for 48 h (probability values)

| | | | | |
|---|---|---|---|---|
| A: 2.5 wt % | 1 | | | |
| cannabidiol | | | | |
| B: 5.0 wt % | 0.021 | 1 | | |
| cannabidiol | | | | |
| C: 2.5 wt % | 0.333 | 0.056 | 1 | |
| cannabidiol | | | | |
| D: 5.0 wt % | 0.027 | 0.820 | 0.080 | 1 |
| cannabidiol | | | | |

T Test for Epidermis (probability values)

| | | | | |
|---|---|---|---|---|
| A: 2.5 wt % | 1 | | | |
| cannabidiol | | | | |
| B: 5.0 wt % | 0.013 | 1 | | |
| cannabidiol | | | | |
| C: 2.5 wt % | 0.745 | 0.008 | 1 | |
| cannabidiol | | | | |
| D: 5.0 wt % | 0.062 | 0.201 | 0.035 | 1 |
| cannabidiol | | | | |

T Test for Dermis (probability values)

| | | | | |
|---|---|---|---|---|
| A: 2.5 wt % | 1 | | | |
| cannabidiol | | | | |
| B: 5.0 wt % | 0.492 | 1 | | |
| cannabidiol | | | | |
| C: 2.5 wt % | 0.567 | 0.777 | 1 | |
| cannabidiol | | | | |
| D: 5.0 wt % | 0.763 | 0.263 | 0.227 | 1 |
| cannabidiol | | | | |

Based on the results of the T test analysis, it was observed that A: 2.5 wt % cannabidiol and B: 5.0 wt % cannabidiol were statistically different at 24 and 48 hrs and in the epidermis with greater than 95% confidence (p-values are 0.040, 0.021, and 0.013 respectively). The dermal values for A: 2.5 wt % cannabidiol and B: 5.0 wt % cannabidiol were not statistically different with a p-value of 0.492.

Based on the results of the T test analysis, it was also observed that C: 2.5 wt % cannabidiol and D: 5.0 wt % cannabidiol were statistically different at 24 and 48 hrs and in the epidermis with greater than 90% confidence (p-values are 0.022, 0.080, and 0.035 respectively). The dermal values for C: 2.5 wt % cannabidiol and D: 5.0 wt % cannabidiol were not statistically different with a p-value of 0.227.

Finally, based on the results of the T test analysis, there were no statistically significant differences between A: 2.5 wt % cannabidiol and C: 2.5 wt % cannabidiol or between the B: 5.0 wt % cannabidiol and D: 5.0 wt % cannabidiol. These data suggest that there is not a meaningful difference in the flux parameters between the two different CBD formulations.

From the foregoing Examples, it is expected that the use of cannabinoids, such as cannabidiol in accordance with the present invention can deliver increased amounts of cannabidiol into the epidermis and dermis and be used to treat and/or improve the healing of acne. Generally, treatment in accordance with the present invention will result in a shortened healing time.

1. A pharmaceutical composition comprising a cannabinoid and a siloxane wherein the cannabinoid is dissolved in the composition.

2. The pharmaceutical composition according to claim 1 wherein the cannabinoid is cannabidiol.

3. The pharmaceutical composition according to claim wherein the composition is for topical application.

4. The pharmaceutical composition according to any one of the preceding claims wherein the siloxane
is selected from the group consisting of: hexamethyldisiloxane, octamethyltrisiloxane and combinations thereof.

5. The pharmaceutical composition according to claim 1 further comprising a residual solvent.

6. The pharmaceutical composition according to claim 5 wherein the residual solvent is selected from the group consisting of: alkyl polypropylene glycol/polyethylene glycol ether (alkyl PEG/PPG ether) and/or fatty alcohol.

7. The pharmaceutical composition according to claim 6 wherein the alkyl PEG/PPG ether:
a) has a PEG/PPG chain length of between 10-50 PG units and an ether component of between 2-20 carbons, wherein the sum of the PG units and the carbons of the ether component is between 20 and 60;
b) has a low volatility such that less than 5% would evaporate at skin temperature over 24 hours;
c) is a liquid at about 30° C., or less; and/or
d) is selected from the group consisting of: polypropylene glycol ethers of stearyl alcohol and butyl alcohol.

8. The pharmaceutical composition according to claim 6 wherein the relative amount of alkyl PEG/PPG ether is at least 1% w/w

9. The pharmaceutical composition according to claim 6 wherein the fatty alcohol:
a) has a low volatility such that less than 5% would evaporate at skin temperature over 24 hours;
b) is a C₁₂₋₂₂ fatty alcohol and/or
c) is a liquid at about 30° C., or less.

10. The pharmaceutical composition according to claim 9 wherein the fatty alcohol is selected from the group consisting of: oleyl alcohol, isostearyl alcohol, octyldodecyl alcohol, and 2-hexyl decyl alcohol.

11. The pharmaceutical composition according to claim 1 further comprising a low molecular weight alcohol.

12. The pharmaceutical composition according to claim 11 wherein the low molecular weight alcohol:
i. is a liquid at ambient temperatures;
ii. has a level of volatility about the same as that of isopropyl alcohol; and/or
iii. is selected from the group consisting of: C₂₋₆ alcohols, and combinations thereof.

13. The pharmaceutical composition according to claim 12 wherein the alcohol is selected from the group consisting of: ethyl alcohol, n-propanol, isopropyl alcohol and combinations thereof.

14. The pharmaceutical composition according to claim 1, wherein the concentration of cannabinoid in the topical composition is selected from the group consisting of: at least 2% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w and at least 15% w/w.

15. The pharmaceutical composition according to claim 1, wherein concentration of cannabinoid in the topical composition is selected from the group consisting of: at least 20% w/w, at least 30% w/w, at least 40% w/w, at least 50% w/w, at least 60% w/w, at least 70% w/w, at least 80% w/w, at least 90% w/w, at least 95% w/w, and at least 99% w/w.

16. A method for treating acne in a patient in need of such treatment, the method comprising topically administering a prophylactically or therapeutically effective amount of a pharmaceutical composition according to any one of the preceding claims.

17. (canceled)

18. (canceled)
